(19) United States
(12) Patent Application Publication
(10) Pub. No.: US 2019/0134308 A1
(43) Pub. Date: May 9, 2019

(54) SYSTEM AND METHOD FOR MONITORING BLOOD CELL LEVELS IN BLOOD FLOW USING PPG TECHNOLOGY

(71) Applicant: Sanmina Corporation, San Jose, CA (US)

(72) Inventor: Robert Steven Newberry, New Hope, AL (US)

(21) Appl. No.: 16/239,417

(22) Filed: Jan. 3, 2019

Related U.S. Application Data
(63) Continuation-in-part of application No. 14/866,500, filed on Sep. 25, 2015.
Application No. 14/866,500 is a continuation-in-part of application No. 15/811,479, filed on Nov. 13, 2017, now Pat. No. 10,238,346.
Application No. 15/811,479 is a continuation-in-part of application No. 15/485,816, filed on Apr. 12, 2017, now Pat. No. 10,155,087.
Application No. 15/485,816 is a continuation of application No. 15/276,760, filed on Sep. 26, 2016, now Pat. No. 9,636,457.
Application No. 15/276,760 is a continuation-in-part of application No. 15/718,721, filed on Sep. 28, 2017.
Application No. 15/718,721 is a continuation of application No. 15/622,941, filed on Jun. 14, 2017, now Pat. No. 9,788,767.
Application No. 15/622,941 is a continuation-in-part of application No. 15/804,581, filed on Nov. 6, 2017, now Pat. No. 10,231,674.
Application No. 15/804,581 is a continuation of application No. 15/404,117, filed on Jan. 11, 2017.
Application No. 15/404,117 is a continuation-in-part of application No. 15/958,620, filed on Apr. 20, 2018.
Application No. 15/958,620 is a continuation of application No. 15/680,991, filed on Aug. 18, 2017, now Pat. No. 9,968,289.
Application No. 15/680,991 is a continuation-in-part of application No. 15/400,916, filed on Jan. 6, 2017.
Application No. 15/400,916 is a continuation-in-part of application No. 16/019,518, filed on Jun. 26, 2018.
Application No. 16/019,518 is a division of application No. 15/867,632, filed on Jan. 10, 2018, now Pat. No. 10,039,500.
Application No. 15/867,632 is a continuation-in-part of application No. 15/859,147, filed on Dec. 29, 2017, now Pat. No. 10,194,871.
Application No. 15/859,147 is a continuation-in-part of application No. 15/898,580, filed on Feb. 17, 2018.
(60) Provisional application No. 62/613,388, filed on Jan. 3, 2018.
(60) Provisional application No. 62/675,151, filed on May 22, 2018.
(60) Provisional application No. 62/463,104, filed on Feb. 24, 2017.

(51) Int. Cl. A61M 5/172; A61M 5/32; A61M 5/142

(52) U.S. Cl. CPC A61M 5/1723; A61M 5/3298; A61M 5/14248; A61M 2205/8206; A61M 2205/8268; A61M 2205/3306; A61M 5/145; A61M 2205/3584; A61M 2230/63; A61M 2230/06; A61M 2230/30; A61M 2230/42; A61M 2230/20

(57) ABSTRACT

A biosensor includes a photoplethysmography (PPG) circuit configured to obtain spectral responses at one or more wavelengths from skin tissue of the patient over a predetermined time period. The biosensor monitors the color of the blood to detect a change in the baseline color of blood flow over the predetermined time period. The biosensor may determine a level of red blood cells or white blood cells or a risk of an infection using the change in the baseline color of blood flow. The biosensor may also detect other parameters to detect an infection or identify a type of infection including liver enzyme levels, nitric oxide levels, heart rate or vasodilation in the underlying tissue.

### CROSS REFERENCE TO RELATED APPLICATIONS

The present application claims priority under 35 U.S.C. § 119(e) to U.S. Provisional Application No. 62/613,388 entitled, “SYSTEM AND METHOD FOR INFECTION DISCRIMINATION USING PPG TECHNOLOGY,” filed Jan. 3, 2018, and hereby expressly incorporated by reference herein.

The present application claims priority under 35 U.S.C. § 119(e) to U.S. Provisional Application No. 62/675,151 entitled, “SYSTEM AND METHOD OF A BIOSENSOR FOR DETECTION OF VASODILATION,” filed May 22, 2018, and hereby expressly incorporated by reference herein.

The present application claims priority under 35 U.S.C. § 120 as a continuation in part to U.S. patent application Ser. No. 14/866,500 entitled, “SYSTEM AND METHOD FOR GLUCOSE MONITORING,” filed Sep. 25, 2015, and hereby expressly incorporated by reference herein.

The present application claims priority under 35 U.S.C. § 120 as a continuation in part application to U.S. patent application Ser. No. 15/811,479 entitled, “SYSTEM AND METHOD FOR A BIOSENSOR INTEGRATED IN A VEHICLE,” filed November 13, 2017 and hereby expressly incorporated by reference herein.

The present application claims priority under 35 U.S.C. § 120 as a continuation in part application to U.S. patent application Ser. No. 15/485,816 entitled, “SYSTEM AND METHOD FOR A DRUG DELIVERY AND BIOSENSOR PATCH,” filed Apr. 12, 2017 and hereby expressly incorporated by reference herein, which claims priority under 35 U.S.C. § 120 as a continuation application to U.S. Utility application Ser. No. 15/276,760, entitled, “SYSTEM AND METHOD FOR A DRUG DELIVERY AND BIOSENSOR PATCH,” filed Sep. 26, 2016, now U.S. Pat. No. 9,636,457 issued May 2, 2017, which is hereby expressly incorporated by reference herein.

The present application claims priority under 35 U.S.C. § 120 as a continuation in part application to U.S. patent application Ser. No. 15/718,721 entitled, “SYSTEM AND METHOD FOR MONITORING NITRIC OXIDE LEVELS USING A NON-INVASIVE, MULTI-BAND BIOSENSOR,” filed Sep. 28, 2017 and hereby expressly incorporated by reference herein, which claims priority as a continuation application to U.S. Utility application Ser. No. 15/622,941 entitled, “SYSTEM AND METHOD FOR MONITORING NITRIC OXIDE LEVELS USING A NON-INVASIVE, MULTI-BAND BIOSENSOR,” filed Jun. 14, 2017, now U.S. Pat. No. 9,788,767 issued October 17, 2017, and hereby expressly incorporated by reference herein, which claims priority under 35 U.S.C. § 119 to U.S. Provisional Application No. 62/463,104 entitled, “SYSTEM AND METHOD FOR MONITORING NITRIC OXIDE LEVELS USING A NON-INVASIVE, MULTI-BAND BIOSENSOR,” filed Feb. 24, 2017, and hereby expressly incorporated by reference herein.

The present application claims priority under 35 U.S.C. § 120 as a continuation in part application to U.S. patent application Ser. No. 15/804,581 entitled, “SYSTEM AND METHOD FOR HEALTH MONITORING INCLUDING A USER DEVICE AND BIOSENSOR,” filed Nov. 6, 2017 and hereby expressly incorporated by reference herein, which claims priority as a continuation application to U.S. patent application Ser. No. 15/404,117 entitled, “SYSTEM AND METHOD FOR HEALTH MONITORING INCLUDING A USER DEVICE AND BIOSENSOR,” filed Jan. 11, 2017 and hereby expressly incorporated by reference herein.

The present application claims priority under 35 U.S.C. § 120 as a continuation in part application to U.S. Utility application Ser. No. 15/958,620 entitled, “SYSTEM AND METHOD FOR DETECTING A HEALTH CONDITION USING AN OPTICAL SENSOR,” filed Apr. 20, 2018, and hereby expressly incorporated by reference herein which claims priority under 35 U.S.C. § 120 as a continuation application to U.S. Utility application Ser. No. 15/680,991 entitled, “SYSTEM AND METHOD FOR DETECTING A SEPSIS CONDITION,” filed Aug. 18, 2017, and hereby expressly incorporated by reference herein and now issued as U.S. Pat. No. 9,968,289 issued May 15, 2018.

The present application claims priority under 35 U.S.C. § 120 as a continuation in part application to U.S. patent application Ser. No. 15/400,916 entitled, “SYSTEM AND METHOD FOR HEALTH MONITORING INCLUDING A REMOTE DEVICE,” filed Jan. 6, 2017 and hereby expressly incorporated by reference herein.

The present application claims priority under 35 U.S.C. § 120 as a continuation in part to U.S. patent application Ser. No. 16/019,518 entitled, “SYSTEM AND METHOD FOR BLOOD TYPING USING PPG TECHNOLOGY,” filed Jun. 26, 2018, and hereby expressly incorporated by reference herein, which claims priority under 35 U.S.C. § 120 as a divisional to U.S. patent application Ser. No. 15/867,632 entitled, “SYSTEM AND METHOD FOR BLOOD TYPING USING PPG TECHNOLOGY,” filed Jan. 10, 2018, and hereby expressly incorporated by reference herein, and now issued as U.S. Pat. No. 10,039,500 issued Aug. 7, 2018.

The present application claims priority under 35 U.S.C. § 120 as a continuation in part to U.S. Patent Application 15/859,147 entitled, “VEHICLULAR HEALTH MONITORING SYSTEM AND METHOD,” filed December 29, 2017 and hereby expressly incorporated by reference herein.

The present application claims priority under 35 U.S.C. § 120 as a continuation in part to U.S. patent application Ser. No. 15/898,580 entitled, “SYSTEM AND METHOD FOR OBTAINING HEALTH DATA USING A NEURAL NETWORK,” filed Feb. 17, 2018, and hereby expressly incorporated by reference herein.

### FIELD

This application relates to a system and methods of non-invasive, autonomous health monitoring, and in particular a system and method for monitoring levels of white blood cells or red blood cells in blood flow using optical sensor technology.

### BACKGROUND

Due the rising trends of care induced infections, a quick and simpler determination of viral growth within the body would be very practical from a care perspective to reduce the amount of time and money currently spent on blood sampling methods.

For example, two types of infections include sepsis and staph infections. Staph infections are caused by staphylococcus bacteria, including, e.g., Hemolytic and Non-Hemolytic varieties. Sepsis infections include, e.g., Febrile or AFebrile Sepsis. To discern between different types of infections and viruses, various invasive methods have been developed for testing using one or more types of techniques to remove cells from various types of bodily fluids. The methods usually require drawing blood from a blood vessel using a needle and syringe. The blood sample is then transported to a lab for analysis in vitro to determine various levels of substances using physical or chemical measurements.

These known in vitro measurements have disadvantages. The process of obtaining blood samples is time consuming, inconvenient and painful to a patient. It may also disrupt sleep of the patient. The measurements using blood testing is not continuous and may only be updated by taking another blood sample.

One current non-invasive method is known for measuring oxygen saturation in blood vessels using pulse oximeters. Pulse oximeters detect oxygen saturation of hemoglobin by using, e.g., spectrophotometry to determine spectral absorbencies and determining concentration levels of oxygen based on Beer-Lambert law principles. For example, pulse oximetry may use photoplethysmography (PPG) methods for the assessment of oxygen saturation in pulsatile arterial blood flow. The subject's skin at a ‘measurement location’ is illuminated with two distinct wavelengths of light and the relative absorbance at each of the wavelengths is determined. For example, a wavelength in the visible red spectrum (for example, at 660 nm) has an extinction coefficient of hemoglobin that exceeds the extinction coefficient of oxihemoglobin. At a wavelength in the near infrared spectrum (for example, at 940 nm), the extinction coefficient of oxihemoglobin exceeds the extinction coefficient of hemoglobin. The pulse oximeter filters the absorbance of the pulsatile fraction of the blood, i.e. that due to arterial blood (AC components), from the constant absorbance by nonpulsatile venous or capillary blood and other tissue pigments (DC components), to eliminate the effect of tissue absorbance to measure the oxygen saturation of arterial blood.

A practical application of this technique is pulse oximetry, which utilizes a noninvasive sensor to measure oxygen saturation (SpO₂) and pulse rate and can output representative plethysmographic waveforms. Such PPG techniques are heretofore been limited to determining oxygen saturation using wavelengths in the visible red and near infrared spectrum.

As such, there is a need for a system and method that includes a continuous and non-invasive biosensor configured for monitoring levels of white blood cells and/or red blood cells in blood flow in vivo.

In addition, there is a need for a system and method that includes a continuous and non-invasive biosensor configured for monitoring blood flow to detect an infection and a source of the infection in vivo.

### SUMMARY

According to a first aspect, a biosensor includes a photoplethysmography (PPG) circuit configured to obtain spectral responses at one or more wavelengths from skin tissue of a patient; and a processing circuit. The processing circuit is configured to obtain a blood type of the patient; obtain a baseline color of blood flow of the patient using the blood type of the patient; determine a color of the blood flow of the patient using one or more of the spectral responses; and determine a difference in color of the blood flow from the baseline color of the blood flow.

According to a second aspect, a biosensor includes a PPG circuit configured to obtain spectral responses at one or more wavelengths from skin tissue of a patient; and a processing circuit configured to obtain a measurement of a color of blood flow using one or more of the spectral responses. The processing circuit is further configured to compare the measurement of the color of blood flow to a predetermined threshold and generate an indication of a level of white blood cells or red blood cells in blood flow based on the comparison.

According to a third aspect, a biosensor includes a PPG circuit configured to obtain spectral responses at a plurality of wavelengths from skin tissue of the patient; and a processing circuit configured to determine a baseline color of blood flow of the patient using one or more of the plurality of spectral responses. The processing circuit is further configured to determine a change in the baseline color of blood flow using the one or more of the plurality of spectral responses and determine a level in blood flow of at least one of: red blood cells and white blood cells.

According to one or more of the above aspects, the processing circuit determines a change in a level of white blood cells or red blood cells in blood flow using the difference in color of the blood flow from the baseline color of the blood flow.

According to one or more of the above aspects, the processing circuit determines an infection using the difference in the color of the blood flow.

According to one or more of the above aspects, the processing circuit compares the difference in the color of the blood flow to a predetermined threshold and determine a presence of hemolysis in the blood flow based on the comparison.

According to one or more of the above aspects, the processing circuit determines a level of red blood cells affected by hemolysis in the blood flow using the difference in the color of the blood flow.

According to one or more of the above aspects, the processing circuit determines a measurement of a liver enzyme in the blood flow using one or more of the spectral responses and determine a type of infection using the measurement of the liver enzyme and the difference in the color of the blood flow.

According to one or more of the above aspects, the processing circuit determines a measurement of nitric oxide (NO) in the blood flow using one or more of the spectral responses and determine a type of infection using the measurement of NO and the change in the color of the blood flow.

According to one or more of the above aspects, the processing circuit determines a heart rate and vasodilation using one or more of the spectral responses and determine the type of infection using the measurement of the heart rate, vasodilation and the change in the color of the blood flow.

According to one or more of the above aspects, the processing circuit obtains the blood type of the patient by determining a first ratio R value using a first spectral response and a second spectral response of the spectral responses, wherein the first ratio R value is determined from a ratio of alternating current (AC) signal components in the first spectral response and the second spectral response and varies based on one or more of a plurality of types of antigens on surfaces of red blood cells in a blood stream of the user; accessing a calibration database stored in a memory that associates predetermined ratio R values to the plurality of types of antigens on the surfaces of red blood cells; and identifying the blood type of the patient using the first ratio R value and the calibration database.

According to one or more of the above aspects, the biosensor is implemented on a disposable patch including a visible or audible indicator of an infection.

According to one or more of the above aspects, the processing circuit determines a pattern of the one or more of the spectral responses indicating a presence of white blood cells and determines the level of white blood cells in the blood flow using the pattern of the one or more of the spectral responses.

According to one or more of the above aspects, the processing circuit determines the pattern of the one or more of the spectral responses indicating a presence of white blood cells by determining a change in the width and shape of the spectral response due to a size of the white blood cells.

According to one or more of the above aspects, the processing circuit determines an increase in concentration of neutrophil white blood cells in response to the color of the blood flow and the change in the pattern of the one or more of the spectral responses.

According to one or more of the above aspects, the type of infection includes: afebrile sepsis, febrile sepsis, hemolytic staph infection or non-hemolytic staph infection.

### BRIEF DESCRIPTION OF THE DRAWINGS

FIG. 1 illustrates a schematic block diagram of exemplary components in an embodiment of the biosensor.

FIG. 2 illustrates a schematic block diagram of an embodiment of the PPG circuit in more detail.

FIG. 3 illustrates a logical flow diagram of an embodiment of a method for determining concentration level of a substance in blood flow using Beer-Lambert principles.

FIG. 4 illustrates the spectral response obtained at the plurality of wavelengths with the systolic points and diastolic points aligned over a cardiac cycle.

FIG. 5 illustrates a logical flow diagram of an embodiment of a method of the biosensor.

FIG. 6 illustrates a logical flow diagram of an exemplary method to determine levels of a substance in blood flow using the PPG signals at a plurality of wavelengths.

FIG. 7 illustrates a logical flow diagram of an exemplary method to determine levels of a substance using the spectral responses at a plurality of wavelengths in more detail.

FIG. 8 illustrates a logical flow diagram of an exemplary embodiment of a method for measuring a concentration level of a substance in vivo using shifts in absorbance spectra.

FIG. 9 illustrates a schematic drawing of an exemplary embodiment of a spectral response obtained using an embodiment of the biosensor.

FIG. 10 illustrates a schematic drawing of an exemplary embodiment of results of R values determined using a plurality of methods.

FIG. 11A illustrates a schematic drawing of an exemplary embodiment of an empirical calibration curve for correlating oxygen saturation levels (SpO₂) with R values.

FIG. 11B illustrates a schematic drawing of an exemplary embodiment of an empirical calibration curve for correlating NO levels with R values.

FIG. 12 illustrates a schematic block diagram of an embodiment of a calibration database.

FIG. 13A illustrates a perspective front view of the biosensor in a patch form factor.

FIG. 13B illustrates a perspective back view of the biosensor in a patch form factor.

FIG. 14 illustrates a schematic block diagram of an embodiment of predetermined thresholds of Nitric Oxide (NO) measurements for detecting a risk of sepsis.

FIG. 15 illustrates a logical flow diagram of an embodiment of a method for determining predetermined thresholds for health alert indicators for sepsis.

FIG. 16 illustrates a logical flow diagram of an exemplary embodiment of a method 1600 for identifying Febrile or AFebrile Sepsis by the biosensor.

FIG. 17A illustrates a graphical diagram of an embodiment of clinical data for NO measurements of a healthy person.

FIG. 17B illustrates a graphical diagram of an embodiment of clinical data for NO measurements of a patient with sepsis.

FIG. 18 illustrates a logical flow diagram of a method for detection of hemolysis.

FIG. 19 illustrates a logical flow diagram of a method for detecting a staph infection and identifying a type of staph infection.

FIG. 20 illustrates a logical flow diagram of a method 2000 for detection of an infection and identification of a type of infection.

FIG. 21 illustrates a schematic drawing of an embodiment of a calibration table 2100 for blood groups.

FIG. 22 illustrates a schematic graph of an embodiment of predetermined signal quality parameters for obtaining a blood type in a patient.

FIG. 23 illustrates an exemplary graph of spectral responses of a plurality of wavelengths from clinical data using the biosensor.

FIG. 24 illustrates a logical flow diagram of a method for detecting a presence of infection using a level of white blood cells in blood flow.

FIG. 25A illustrates a schematic diagram of graphs of PPG signals detected from normal tissue.

FIG. 25B illustrates a schematic diagram of graphs of PPG signals detected from tissue after an impact.

FIG. 26A illustrates a schematic diagram of a phasor relationship between PPG signals detected from normal tissue.

FIG. 26B illustrates a schematic diagram of graphs of PPG signals detected from tissue after an impact.

FIG. 26C illustrates a schematic diagram of graphs of PPG signals detected from tissue healing after an impact.

FIG. 27A illustrates a schematic diagram of a phasor relationship of PPG signals detected from normal tissue.

FIG. 27B illustrates a schematic diagram of a phasor relationship of PPG signals detected from tissue at sub-normal temperature.

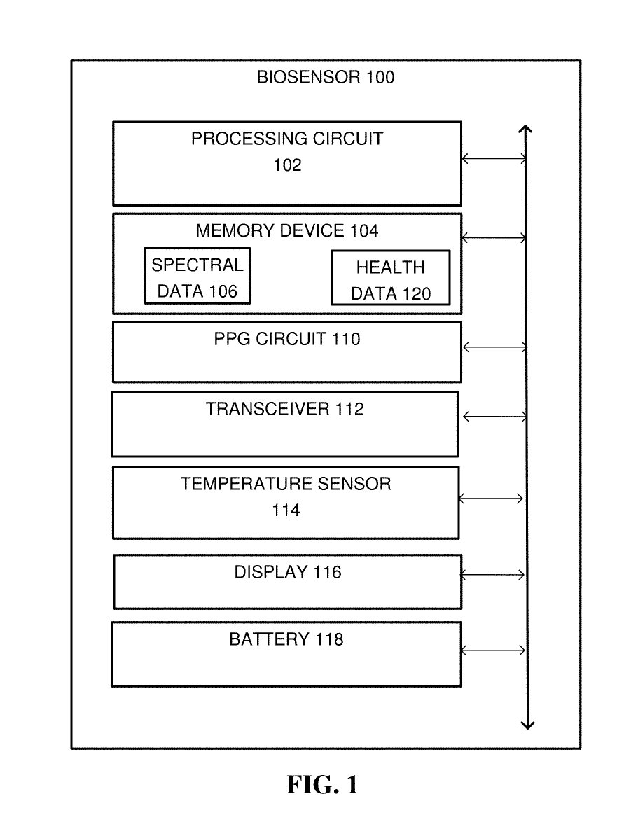

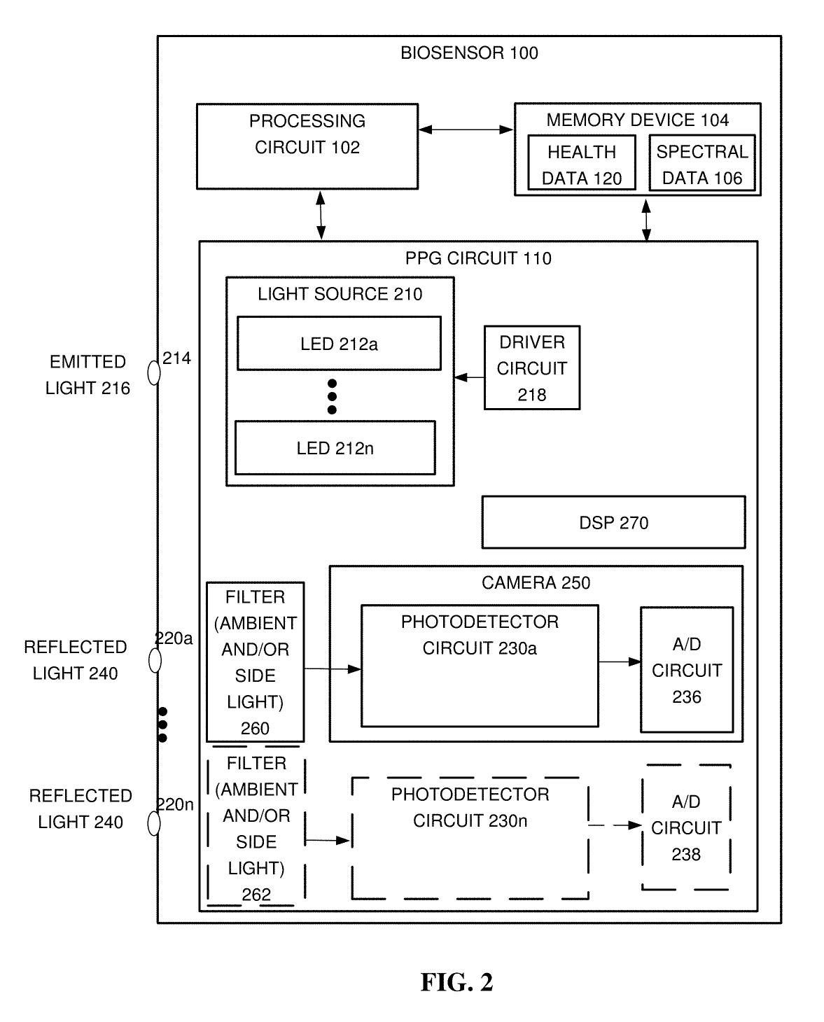

### DETAILED DESCRIPTION

The word “exemplary” or “embodiment” is used herein to mean “serving as an example, instance, or illustration.” Any implementation or aspect described herein as “exemplary” or as an “embodiment” is not necessarily to be construed as preferred or advantageous over other aspects of the disclosure. Likewise, the term “aspects” does not require that all aspects of the disclosure include the discussed feature, advantage, or mode of operation.

Embodiments will now be described in detail with reference to the accompanying drawings. In the following description, numerous specific details are set forth in order to provide a thorough understanding of the aspects described herein. It will be apparent, however, to one skilled in the art, that these and other aspects may be practiced without some or all of these specific details. In addition, well known steps in a method of a process may be omitted from flow diagrams presented herein in order not to obscure the aspects of the disclosure. Similarly, well known components in a device may be omitted from figures and descriptions thereof presented herein in order not to obscure the aspects of the disclosure.

### Overview

A biosensor with PPG technology may measure a level of white blood cells or red blood cells in blood flow. The biosensor may use the level with one or more other parameters to diagnose a presence of an infection and identify a type of infection in vivo in a patient. The biosensor may be used with a human or non-human patient or user, such as a person or animal. With the use of a multi-spectrum PPG sensor it is possible to determine a type of active infection within a body. Due the rising trends of care induced infections, a quick and simpler determination of infection within the body would be very practical from a care perspective to reduce the amount of time and money currently expended on blood sampling methods.

The biosensor detects biomarkers in blood flow to detect changes in levels of white blood cells or red blood cells. The biosensor may detect other substance in blood flow and vital signs to support early clinical detection of an infection and provide identification of a source and/or type of infection. Using different optical wavelengths and processes, the biosensor may detect the presence of antigens of the red blood cells using PPG technology. This enables the diagnosis of the blood type of a user non-invasively. Additionally, the biosensor may measure the skin temperature of a user, heart rate, respiration rate, liver enzyme levels, Nitric Oxide (NO) levels, localized vasodilation, Hemoglobin Ratios, SpO₂ levels, and other blood substance levels. With this additional information available, the biosensor may perform quick, non-invasive PPG testing methods to augment traditional invasive blood laboratory tests for patient care needs.

For example, the biosensor detects common infection types such as, Hemoltic and Nonhemoltic Staph infections, Febrile and Afebrile Sepsis infections, Bacteremia or pneumonia. These types of infections may arise from device contamination in orthopedic implants, sub-clinical infections, care facility acquired or wound infections. Due to the rapid assessment methods available using this technology, a care giver may direct care needs within minutes to provide specific treatments best suitable for a patient without requiring an extensive battery of invasive blood tests to be performed requiring a longer period of time. The biosensor thus enables a physician to more quickly determine critical and timely treatments for conditions like infections.

### Embodiment of the Biosensor

In an embodiment, the driver circuit 218 is configured to control the one or more LEDs 212a-n to generate light at one or more frequencies for predetermined periods of time. The driver circuit 218 may control the LEDs 212a-n to operate concurrently or consecutively. The driver circuit 218 is configured to control a power level, emission period and frequency of emission of the LEDs 212a-n. The driver circuit 218 may also tune a wavelength output of the LEDs212a-n in response to a temperature or other feedback. The biosensor 100 is thus configured to emit one or more wavelengths of light in one or more spectrums that is directed at the surface or epidermal layer of the skin tissue of a user. The emitted light 216 passes through at least one aperture 214 and towards the surface or epidermal layer of the skin tissue of a user.

The PPG circuit 110 further includes one or more photodetector circuits 230a-n. The photodetector circuits 230 may be implemented as part of a camera 250. For example, a first photodetector circuit 230 may be configured to detect visible light and the second photodetector circuit 230 may be configured to detect IR light. Alternatively, a single photodetector 230 may be implemented to detect light across multiple spectrums. When multiple photodetectors 230 are implemented, the detected signals obtained from each of the photodetectors may be added or averaged. Alternatively, a detected light signal with more optimal signal to noise ration may be selected from the multiple photodetector circuits 230a-n.

The first photodetector circuit 230a and the second photodetector circuit 230n may also include a first filter 260 and a second filter 262 configured to filter ambient light and/or scattered light. For example, in some embodiments, only light reflected at an approximately perpendicular angle to the skin surface of the user is desired to pass through the filters. The first photodetector circuit 230a and the second photodetector circuit 230n are coupled to a first analog to digital (A/D) circuit 236 and a second A/D circuit 238. Alternatively, a single A/D circuit may be coupled to each of the photodetector circuits 230a-n. The A/D circuits convert the spectral responses to digital spectral data for processing by a DSP or other processing circuit.

The one or more photodetector circuits 230a-n include one or more types of spectrometers or photodiodes or other types of light detection circuits configured to detect an intensity of light as a function of wavelength over a time period to obtain a spectral response. In use, the one or more photodetector circuits 230a-n detect the intensity of reflected light 240 from skin tissue of a user that enters one or more apertures 220a-n of the biosensor 100. In another example, the one or more photodetector circuits 230a-n detect the intensity of light due to transmissive absorption (e.g., light transmitted through tissues, such as a fingertip or ear lobe). The one or more photodetector circuits 230a-n then obtain a spectral response (a PPG signal) of the reflected or transmissive light by measuring an intensity of the light at one or more wavelengths over a period of time.

In another embodiment, the light source 210 may include a broad spectrum light source, such as a white light to infrared (IR) or near IR LED, that emits light with wavelengths across multiple spectrums, e.g. from 350 nm to 2500 nm. Broad spectrum light sources with different ranges may be implemented. In an aspect, a broad spectrum light source is implemented with a range across 100 nm wavelengths to 2000 nm range of wavelengths in the visible, IR and/or UV frequencies. For example, a broadband tungsten light source for spectroscopy may be used. The spectral response of the reflected light 240 is then measured across the wavelengths in the broad spectrum, e.g. from 350 nm to 2500 nm, concurrently. In an aspect, a charge coupled device (CCD) spectrometer may be configured in the photodetector circuit 230 to measure the spectral response of the detected light over the broad spectrum.

The PPG circuit 110 may also include a digital signal processing (DSP) circuit 270 that includes signal processing of the digital spectral data. For example, the DSP circuit may determine AC or DC components from the spectral responses (PPG signals) or diastolic and systolic points or other spectral data 106. The spectral data may then be processed by the processing circuit 102 to obtain health data 120 of a user. The spectral data 106 may alternatively or in additionally be transmitted by the biosensor 100 to a central control module for processing to obtain health data 120 of a user. The spectral data 106, PPG signals, etc. may be stored in the memory device 104 of the biosensor 100.

In use, the biosensor 100 performs PPG techniques using the PPG circuit 110 to detect the concentration levels of one or more substances in blood flow. In one aspect, the biosensor 100 receives reflected light or transmissive light from skin tissue to obtain a spectral response. The spectral response includes a spectral curve that illustrates an intensity or power or energy at a frequency or wavelength in a spectral region of the detected light over a period of time. The ratio of the resonance absorption peaks from two different frequencies can be calculated and based on the Beer-Lambert law used to obtain the levels of substances in the blood flow.

For example, one or more of the embodiments of the biosensor 100 described herein is configured to detect a concentration level of one or more substances within blood flow using PPG techniques. For example, the biosensor 100 may detect nitric oxide (NO) concentration levels and correlate the NO concentration level to a blood glucose level. The biosensor 100 may also detect oxygen saturation (SaO2 or SpO2) levels in blood flow. The biosensor may also be configured to detect a liver enzyme cytochrome oxidase (P450) enzyme and correlate the P450 concentration level to a blood alcohol level.

The spectral response of a substance or substances in the arterial blood flow is determined in a controlled environment, so that an absorption coefficient α_{g1} can be obtained at a first light wavelength λ1 and at a second wavelength λ2. According to the Beer-Lambert law, light intensity will decrease logarithmically with path length l (such as through an artery of length l). Assuming then an initial intensity Iᵢₙ of light is passed through a path length l, a concentration C_{g} of a substance may be determined. For example, the concentration Cg may be obtained from the following equations:

At the first wavelength λ₁, I₁=Iᵢₙ₁*10^{-(α}^{_{g1}}^{C}^{_{gw}}^{+α}^{_{w1}}^{C}^{_{w}})*l

At the second wavelength λ₂, I₂=Iᵢₙ₂*10^{-(α}^{_{g2}}^{C}^{_{gw}}^{+α}^{_{w2}}^{C}^{_{w}})*l

wherein:

Iᵢₙ₁ is the intensity of the initial light at λ₁

Iᵢₙ₂ is the intensity of the initial light at λ₂

α_{g1} is the absorption coefficient of the substance in arterial blood at λᵢ

α_{g2} is the absorption coefficient of the substance in arterial blood at λ₂

α_{w1} is the absorption coefficient of arterial blood at λ₁

α_{w2} is the absorption coefficient of arterial blood at λ₂

C_{gw} is the concentration of the substance and arterial blood

C_{w} is the concentration of arterial blood

Then letting R equal:

$$R = \frac{log 10 ( \frac{I 1}{I in 1} )}{log 10 ( \frac{I 2}{I in 2} )}$$

The concentration of the substance Cg may then be equal to:

$$Cg = \frac{Cgw}{Cgw + Cw} = \frac{{α}_{w 2} R - {α}_{w 1}}{( {α}_{w 2} - {α}_{gw 2} ) * R - ( {α}_{w 1} - {α}_{gw 1} )}$$

The biosensor 100 may thus determine the concentration of various substances in arterial blood flow from the Beer-Lambert principles using the spectral responses of at least two different wavelengths.

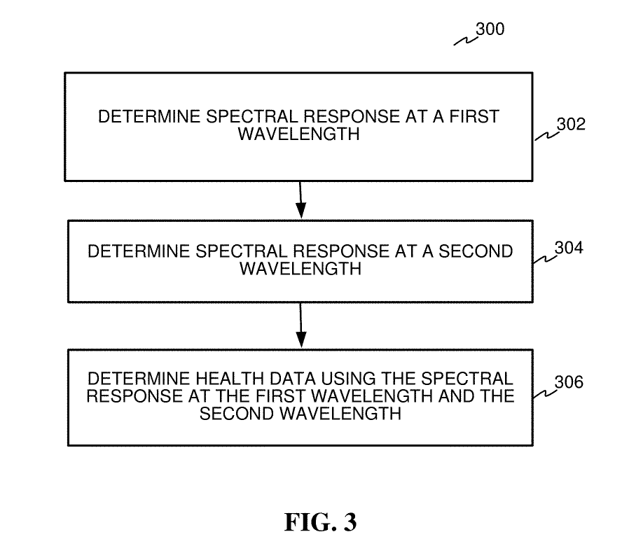

FIG. 3 illustrates a logical flow diagram of an embodiment of a method 300 for determining concentration level of a substance in blood flow using Beer-Lambert principles. The biosensor 100 transmits light at a first predetermined wavelength and at a second predetermined wavelength. The biosensor 100 detects the light (reflected from the skin or transmitted through the skin) and determines the spectral response at the first wavelength at 302 and at the second wavelength at 304. The biosensor 100 then determines health data, such as an indicator or concentration level of substances in blood flow, using the spectral responses of the first and second wavelength at 306. In general, the first predetermined wavelength is selected that has a high absorption coefficient for the substance in blood flow while the second predetermined wavelength is selected that has a lower absorption coefficient for the substance in blood flow. Thus, it is generally desired that the spectral response for the first predetermined wavelength have a higher intensity level in response to the substance than the spectral response for the second predetermined wavelength.

In an embodiment, the biosensor 100 may detect a concentration level of nitric oxide (NO) in blood flow using a first predetermined wavelength in a range of 380-410 nm and in particular at 390 nm or 395 nm. In another aspect, the biosensor 100 may transmit light at the first predetermined wavelength in a range of approximately 1 nm to 50 nm around the first predetermined wavelength. Similarly, the biosensor 100 may transmit light at the second predetermined wavelength in a range of approximately 1 nm to 50 nm around the second predetermined wavelength. The range of wavelengths is determined based on the spectral response since a spectral response may extend over a range of frequencies, not a single frequency (i.e., it has a nonzero linewidth). The light that is reflected or transmitted by NO may spread over a range of wavelengths rather than just the single predetermined wavelength. In addition, the center of the spectral response may be shifted from its nominal central wavelength or the predetermined wavelength. The range of 1 nm to 50 nm is based on the bandwidth of the spectral response line and should include wavelengths with increased light intensity detected for the targeted substance around the predetermined wavelength.

The first spectral response of the light over the first range of wavelengths including the first predetermined wavelength and the second spectral response of the light over the second range of wavelengths including the second predetermined wavelengths is then generated at 302 and 304. The biosensor 100 analyzes the first and second spectral responses to detect an indicator or concentration level of NO in the arterial blood flow at 306. In another embodiment, using absorption coefficients for both Nitric Oxide and Hemoglobin, the concentration of Nitric Oxide can be obtained in arterial blood. A calibration table using human subjects may then correlate amounts of glucose (mG/DL) in relation to R values (NoHb) 404/940 nm.

In another example, the biosensor 100 may also detect vitals, such as heart rate, respiration rate and pulse pressure. The biosensor 100 may also determine a level of vasodilation and a period of vasodilation as described in more detail herein. Because blood flow to the skin can be modulated by multiple other physiological systems, the biosensor 100 may also be used to monitor arterial health, such as hypovolemia or other circulatory conditions.

Photoplethysmography (PPG) is used to measure time-dependent volumetric properties of the pressure pulse wave of blood in blood vessels due to the cardiac cycle. For example, the heartbeat affects the volume of blood flow and the concentration or absorption levels of substances being measured in the blood flow. Over a cardiac cycle, pulsating blood changes the volume of blood flow in a blood vessel. Incident light I_{O} is directed at a tissue site and a certain amount of light is reflected or transmitted and a certain amount of light is absorbed. At a peak of blood flow or volume in a cardiac cycle, the reflected/transmitted light I_{L} is at a minimum due to absorption by the increased blood volume, e.g., due to the pulsating blood in the vessel. At a minimum of blood volume during the cardiac cycle, the transmitted/reflected light I_{H} 416 is at a maximum due to lack of absorption from the pulsating blood.

The biosensor 100 is configured to filter the reflected/transmitted light I_{L} of the pulsating blood from the transmitted/reflected light I_{H}. This filtering isolates the light due to reflection/transmission of the pulsating blood from the light due to reflection/transmission from non-pulsating blood, vessel walls, surrounding tissue, etc. The biosensor 100 may then measure the concentration levels of one or more substances from the reflected/transmitted light I_{L} 814 in the pulsating blood.

For example, incident light I_{O} is directed at a tissue site at one or more wavelengths. The reflected/transmitted light I is detected by a photodetector or sensor array in a camera. At a peak of blood flow or volume, the reflected light I_{L} 414 is at a minimum due to absorption by the pulsating blood, non-pulsating blood, other tissue, etc. At a minimum of blood flow or volume during the cardiac cycle, the Incident or reflected light I_{H} 416 is at a maximum due to lack of absorption from the pulsating blood volume. Since the light I is reflected or traverses through a different volume of blood at the two measurement times, the measurement provided by a PPG sensor is said to be a ‘volumetric measurement’ descriptive of the differential volumes of blood present at a certain location within the user's vessels at different times during the cardiac cycle. These principles described herein may be applied to venous blood flow and arterial blood flow.

In general, the relative magnitudes of the AC and DC contributions to the reflected/transmitted light signal I may be determined. In general, AC contribution of the reflected light signal I is due to the pulsating blood flow. A difference function may thus be computed to determine the relative magnitudes of the AC and DC components of the reflected light I to determine the magnitude of the reflected light due to the pulsating blood flow. The described techniques herein for determining the relative magnitudes of the AC and DC contributions is not intended as limiting. It will be appreciated that other methods may be employed to isolate or otherwise determine the relative magnitude of the light I_{L} due to pulsating blood flow (arterial and/or venous).

In one aspect, the spectral response obtained at each wavelength may be aligned based on the systolic 402 and diastolic 404 points in their respective spectral responses. This alignment is useful to associate each spectral response with a particular stage or phase of the pulse-induced local pressure wave within the blood vessel (which roughly mimics the cardiac cycle 406 and thus include systolic and diastolic stages and sub-stages thereof). This temporal alignment helps to determine the absorption measurements acquired near a systolic point in time of the cardiac cycle and near the diastolic point in time of the cardiac cycle 406 associated with the local pressure wave within the user's blood vessels. This measured local pulse timing information may be useful for properly interpreting the absorption measurements in order to determine the relative contributions of the AC and DC components measured by the biosensor 100. So, for one or more wavelengths, the systolic points 402 and diastolic points 404 in the spectral response are determined. These systolic points 402 and diastolic pointsv404 for the one or more wavelengths may then be aligned as a method to discern concurrent responses across the one or more wavelengths.

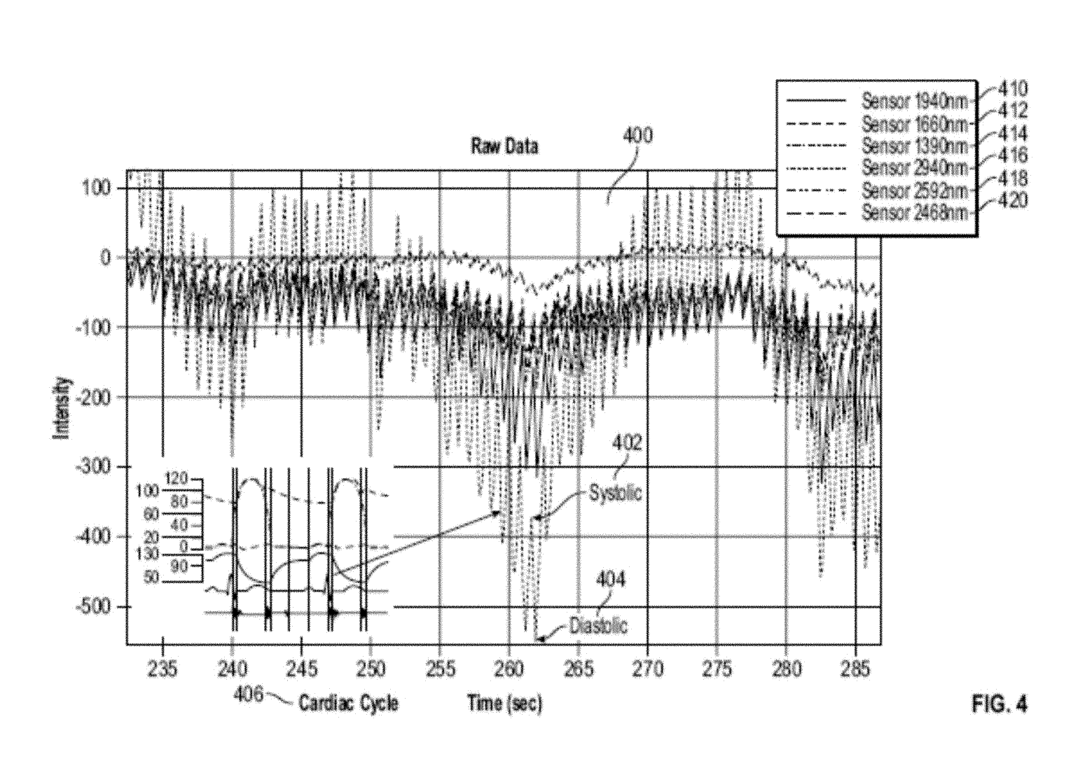

In another embodiment, the systolic points 402 and diastolic points 404 in the absorbance measurements are temporally correlated to the pulse-driven pressure wave within the blood vessels—which may differ from the cardiac cycle. In another embodiment, the biosensor 100 may concurrently measure the intensity reflected at each the plurality of wavelengths. Since the measurements are concurrent, no alignment of the spectral responses of the plurality of wavelengths may be necessary. FIG. 4 illustrates the spectral response obtained at the plurality of wavelengths with the systolic points 402 and diastolic points 404 aligned over a cardiac cycle 406.

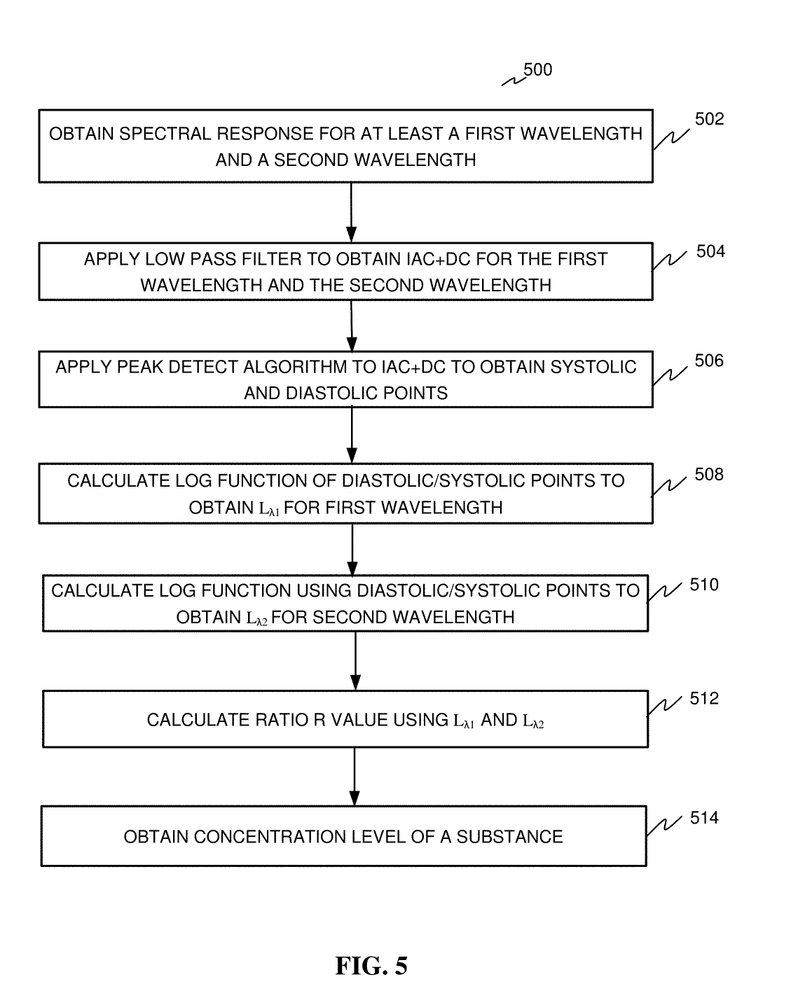

FIG. 5 illustrates a logical flow diagram of an embodiment of a method 500 of the biosensor 100. In one aspect, the biosensor 100 emits and detects light at a plurality of predetermined frequencies or wavelengths, such as approximately 940 nm, 660 nm, 390 nm, 592 nm, and 468 nm or in ranges thereof. The light is pulsed for a predetermined period of time (such as 100 usec or 200 Hz) sequentially or simultaneously at each predetermined wavelength. In another aspect, light may be pulsed in a wavelength range of 1 nm to 50 nm around each of the predetermined wavelengths. For example, for the predetermined wavelength 390 nm, the biosensor 100 may transmit light directed at skin tissue of the user in a range of 360 nm to 410 nm including the predetermined wavelength 390 nm. For the predetermined wavelength of 940 nm, the biosensor 100 may transmit light directed at the skin tissue of the user in a range of 920 nm to 975 nm. In another embodiment, the light is pulsed simultaneously at least at each of the predetermined wavelengths (and in a range around the wavelengths).

The spectral responses are obtained around the plurality of wavelengths, including at least a first wavelength and a second wavelength at 502. The spectral responses may be measured over a predetermined period (such as 300 usec.) or at least over 2-3 cardiac cycles. This measurement process is repeated continuously, e.g., pulsing the light at 10-100 Hz and obtaining spectral responses over a desired measurement period, e.g. from 1-2 seconds to 1-2 minutes or from 2-3 hours to continuously over days or weeks. The spectral data obtained by the PPG circuit 110, such as the digital or analog spectral responses, may be processed locally by the biosensor 100 or transmitted to a central control module for processing.

The systolic and diastolic points of the spectral response are then determined. Because the human pulse is typically on the order of magnitude of one 1 Hz, typically the time differences between the systolic and diastolic points are on the order of magnitude of milliseconds or tens of milliseconds or hundreds of milliseconds. Thus, spectral response measurements may be obtained at a frequency of around 10-100 Hz over the desired measurement period. The spectral responses are obtained over one or more cardiac cycles and systolic and diastolic points of the spectral responses are determined. Preferably, the spectral response is obtained over at least three cardiac cycles in order to obtain a heart rate.

A low pass filter (such as a 5 Hz low pass filter) is applied to the spectral response signal at 504. The relative contributions of the AC and DC components are obtained I_{AC+DC} and I_{AC}. A peak detection algorithm is applied to determine the systolic and diastolic points at 506. If not detected concurrently, the systolic and diastolic points of the spectral response for each of the wavelengths may be aligned or may be aligned with systolic and diastolic points of a pressure pulse waveform or cardiac cycle.

Beer Lambert equations are then applied as described herein. For example, the L_{λ} values are then calculated for the first wavelength λ₁ at 508 and the second wavelength λ₂ at 510, wherein the L_{λ} values for a wavelength equals:

$${L}_{λ} = Log 10 ( \frac{IAC + DC}{IDC} )$$

wherein I_{AC+DC} is the intensity of the detected light with AC and DC components and I_{DC} is the intensity of the detected light with the AC component filtered by the low pass filter. The value L_{λ} isolates the spectral response due to pulsating arterial blood flow, e.g. the AC component of the spectral response.

A ratio R of the L_{λ} values at two wavelengths may then be determined at 512. For example, the ratio R may be obtained from the following:

$$Ratio R = \frac{L λ 1}{L λ 2}$$

The spectral responses may be measured and the L_{λ} values and Ratio R determined continuously, e.g. every 1-2 seconds, and the obtained L_{λ} values and/or Ratio R averaged over a predetermined time period, such as over 1-2 minutes. The concentration level of a substance may then be obtained from the R value and a calibration database at 514. The biosensor 100 may continuously monitor a user over 2-3 hours or continuously over days or weeks.

In one embodiment, the R_{390,940} value with L_{λ1=390} nm and L₂₌₉₄₀ may be non-invasively and quickly and easily obtained using the biosensor 100 to determine a concentration level of nitric oxide NO in blood flow of a user. In particular, in unexpected results, it is believed that the nitric oxide NO levels in the blood flow is being measured at least in part by the biosensor 100 at wavelengths in the range of 380-410 and in particular at λᵢ=390 nm. Thus, the biosensor 100 measurements to determine the L₃₉₀ nm values are the first time NO concentration levels in arterial blood flow have been measured directly in vivo. These and other aspects of the biosensor 100 are described in more detail herein with clinical trial results.

### Embodiment—Determination of Concentration Level of a Substance using PPG Signals at a Plurality of Wavelengths

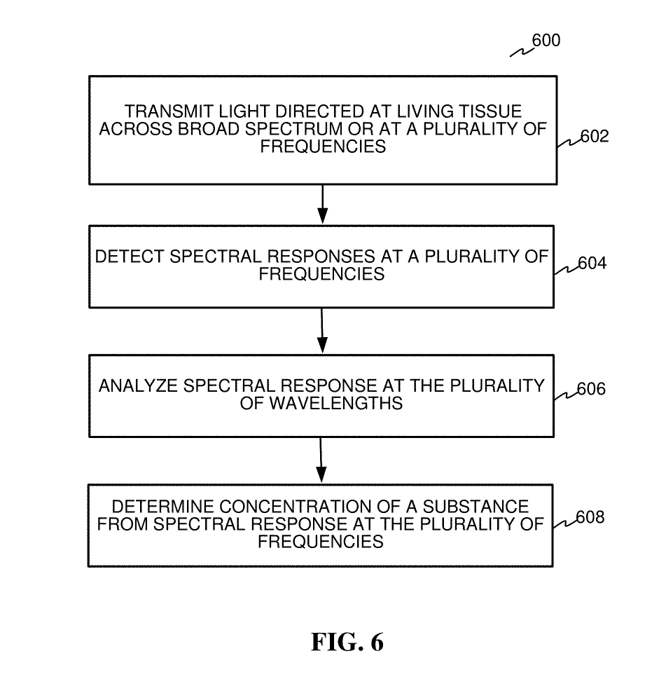

FIG. 6 illustrates a logical flow diagram of an exemplary method 600 to determine levels of a substance in blood flow using the PPG signals at a plurality of wavelengths. The absorption coefficient of a substance may be sufficiently higher at a plurality of wavelengths, e.g. due to isoforms or derivative compounds. For example, the increased intensity of light at a plurality of wavelengths may be due to reflectance by isoforms or other compounds in the arterial blood flow. Another method for determining the concentration levels may then be used by measuring the spectral responses and determining L and R values at a plurality of different wavelengths of light. In this example then, the concentration level of the substance is determined using spectral responses at multiple wavelengths. An example for calculating the concentration of a substance over multiple wavelengths may be performed using a linear function, such as is illustrated herein below.

LN(I₁₋ₙ)=Σᵢ₌₀ⁿμi*Ci

wherein,

I₁₋ₙ=intensity of light at wavelengths λ₁₋ₙ

μₙ=absorption coefficient of substance 1, 2, . . . n at wavelengths λ₁₋ₙ

Cₙ=Concentration level of substance 1, 2, . . . n

When the absorption coefficients μ₁₋ₙ of a substance, its isoforms or other compounds including the substance are known at the wavelengths λ₁₋ₙ, then the concentration level C of the substances may be determined from the spectral responses at the wavelengths λ₁₋ₙ (and e.g., including a range of 1 nm to 50 nm around each of the wavelengths). The concentration level of the substance may be isolated from the isoforms or other compounds by compensating for the concentration of the compounds. Thus, using the spectral responses at multiple frequencies provides a more robust determination of the concentration level of a substance.

In use, the biosensor 100 transmits light directed at skin tissue at a plurality of wavelengths or over a broad spectrum at 602. The spectral response of light from the skin tissue is detected at 604, and the spectral responses are analyzed at a plurality of wavelengths (and in one aspect including a range of +/−10 to 50 nm around each of the wavelengths) at 606. Then, the concentration level C of the substance may be determined using the spectral responses at the plurality of wavelengths at 608. The concentration level of the substance may be isolated from isoforms or other compounds by compensating for the concentration of the compounds. For example, using absorption coefficients for Nitric Oxide and Hemoglobin, the concentration of Nitric Oxide can be obtained in arterial blood. A calibration table using human subjects may then to correlate amounts of glucose (mG/DL) in relation to R values (NoHb) 404/940 nm.

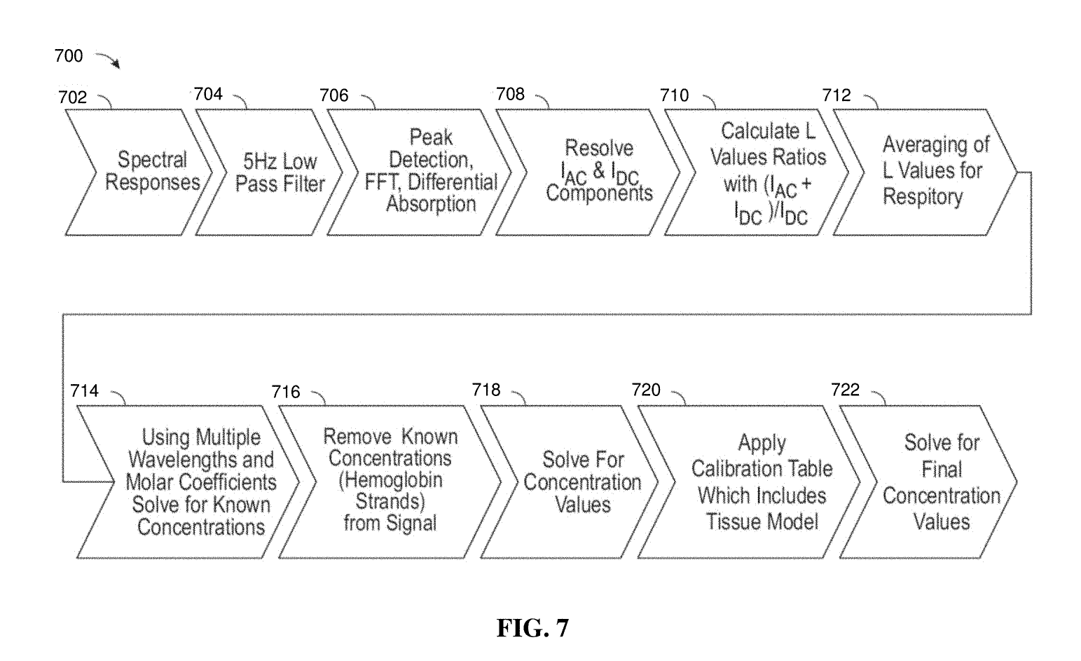

FIG. 7 illustrates a logical flow diagram of an exemplary method 700 to determine levels of a substance using the spectral responses at a plurality of wavelengths in more detail. The spectral responses are obtained at 702. The spectral response signals include AC and DC components I_{AC+DC}. A low pass filter (such as a 5 Hz low pass filter) is applied to each of the spectral response signals I_{AC+DC} to isolate the DC component of each of the spectral response signals I_{DC} at 704. The AC fluctuation is due to the pulsatile expansion of the vessels due to the volume increase in pulsating blood. In order to measure the AC fluctuation, measurements are taken at different times and a peak detection algorithm is used to determine the diastolic point and the systolic point of the spectral responses at 706. A Fast Fourier transform (FFT) algorithm may also be used to isolate the DC component I_{DC} and AC component of each spectral response signal at 706. A differential absorption technique may also be used as described in more detail herein. The I_{DC} component is thus isolated from the spectral signal at 708.

The I_{AC+DC} and I_{DC} components are then used to compute the L values at 710. For example, a logarithmic function may be applied to the ratio of I_{AC+DC} and I_{DC} to obtain an L value for each of the wavelengths L_{λ1-n}. Since the respiratory cycle affects the PPG signals, the L values may be averaged over a respiratory cycle and/or over another predetermined time period (such as over a 1-2 minute time period) or over a plurality of cardiac cycles at 712.

In an embodiment, isoforms of a substance may be attached in the blood stream to one or more types of hemoglobin compounds. The concentration level of the hemoglobin compounds may then need to be accounted for to isolate the concentration level of the substance from the hemoglobin compounds. For example, nitric oxide (NO) is found in the blood stream in a gaseous form and also attached to hemoglobin compounds. Thus, the spectral responses obtained around 390 nm (+/−20 nm) may include a concentration level of the hemoglobin compounds as well as nitric oxide. The hemoglobin compound concentration levels must thus be compensated for to isolate the nitric oxide concentration levels. Multiple wavelengths and absorption coefficients for hemoglobin are used to determine a concentration of the hemoglobin compounds at 714. Other methods may also be used to obtain a concentration level of hemoglobin in the blood flow as well. The concentration of the hemoglobin compounds is then adjusted from the measurements at 716. The concentration values of the substance may then be obtained at 718. For example, the R values are then determined at 718.

To determine a concentration level of the substance, a calibration table or database is used that associates the obtained R value to a concentration level of the substance at 720. The calibration database correlates the R value with a concentration level. The calibration database may be generated for a specific user or may be generated from clinical data of a large sample population. For example, it is determined that the R values should correlate to similar NO concentration levels across a large sample population. Thus, the calibration database may be generated from testing of a large sample of a general population to associate R values and NO concentration levels.

In addition, the R values may vary depending on various factors, such as underlying skin tissue. For example, the R values may vary for spectral responses obtained from an abdominal area versus measurements from a wrist or finger due to the varying tissue characteristics. The calibration database may thus provide different correlations between the R values and concentration levels of a substance depending on the underlying skin tissue characteristics. The concentration level of the substance in blood flow is then obtained using the calibration table at 722. The concentration level may be expressed as mmol/liter, as a saturation level percentage, as a relative level on a scale, etc.

### Embodiment—Determination of Concentration Levels of a Substance using Shifts in Absorbance Peaks

In another embodiment, a concentration level of a substance may be obtained from measuring a characteristic shift in an absorbance peak of hemoglobin. For example, the absorbance peak for methemoglobin shifts from around 433 nm to 406 nm in the presence of NO. The advantage of the measurement of NO by monitoring methemoglobin production includes the wide availability of spectrophotometers, avoidance of sample acidification, and the relative stability of methemoglobin. Furthermore, as the reduced hemoglobin is present from the beginning of an experiment, NO synthesis can be measured continuously, removing the uncertainty as to when to sample for NO.

The biosensor 100 may detect nitric oxide in vivo using PPG techniques by measuring the shift in the absorbance spectra curve of reduced hemoglobin in tissue and/or arterial blood flow. The absorbance spectra curve shifts with a peak from around 430 nm to a peak around 411 nm depending on the production of methemoglobin. The greater the degree of the shift of the peak of the curve, the higher the production of methemoglobin and NO concentration level. Correlations may be determined between the degree of the measured shift in the absorbance spectra curve of reduced hemoglobin to an NO concentration level. The correlations may be determined from a large sample population or for a particular user and stored in a calibration database. The biosensor 100 may thus obtain an NO concentration level by measuring the shift of the absorbance spectra curve of reduced hemoglobin. A similar method of determining shifts in absorbance spectra may be implemented to determine a blood concentration level of other substances.

The biosensor 100 may obtain an NO concentration level by measuring the shift of the absorbance spectra curve of deoxygenated hemoglobin and/or by measuring the shift of the absorbance spectra curve of oxygenated hemoglobin in vivo. The biosensor 100 may then access a calibration database that correlates the measured shift in the absorbance spectra curve of deoxygenated hemoglobin to an NO concentration level. Similarly, the biosensor may access a calibration database that correlates the measured shift in the absorbance spectra curve of oxygenated hemoglobin to an NO concentration level.

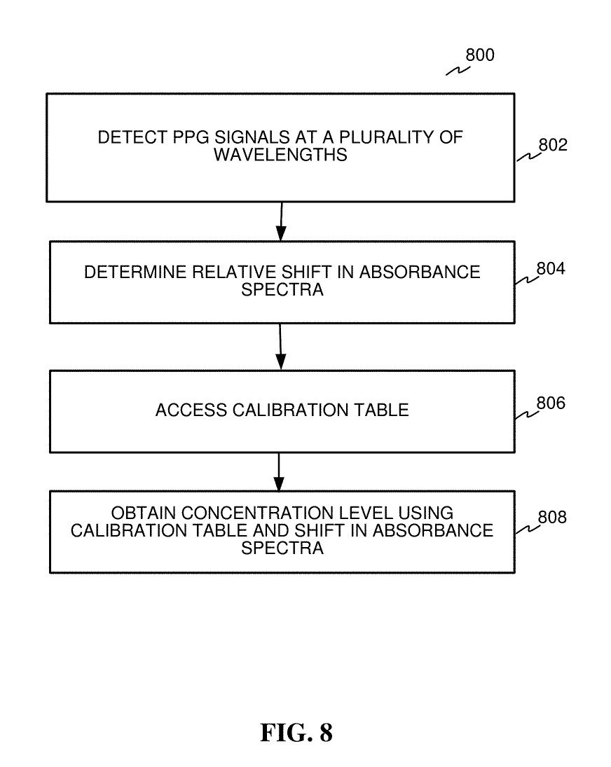

FIG. 8 illustrates a logical flow diagram of an exemplary embodiment of a method 800 for measuring a concentration level of a substance in vivo using shifts in absorbance spectra. The biosensor 100 may obtain a concentration of the substance by measuring shifts in absorbance spectra of one or more substances that interact with the substance. For example, the one or more substances may include oxygenated and deoxygenated hemoglobin (HB). The PPG circuit 110 detects PPG signals at a plurality of wavelengths with a high absorption coefficient of the one or more substances that interact with the substance at 802. The biosensor 100 determines the relative shift in the absorbance spectra for the substance at 804. For example, the biosensor 100 may measure the absorbance spectra curve of deoxygenated HB and determine its relative shift or peak between the range of approximately 430 nm and 405 nm. In another example, the biosensor 100 may measure the absorbance spectra curve of oxygenated HB and determine its relative shift or peak between 421 nm and 393 nm.

The biosensor 100 accesses a calibration database that correlates the relative shift in the absorbance spectra of the substance with a concentration level of the substance at 806. The biosensor 100 may thus obtain a concentration level of the substance in blood flow using a calibration database and the measured relative shift in absorbance spectra at 808.

The various methods thus include one or more of: Peak & Valley (e.g., peak detection), FFT, and differential absorption. Each of the methods require different amounts of computational time which affects overall embedded computing time for each signal, and therefore can be optimized and selectively validated with empirical data through large clinical sample studies. The biosensor 100 may use a plurality of these methods to determine a plurality of values for the concentration level of the substance. The biosensor 100 may determine a final concentration value using the plurality of values. For example, the biosensor 100 may average the values, obtain a mean of the values, etc.

The biosensor 100 may be configured for measurement on a fingertip or palm, wrist, an arm, forehead, chest, abdominal area, ear lobe, or other area of the skin or body or living tissue. The characteristics of underlying tissue vary depending on the area of the body, e.g. the underlying tissue of an abdominal area has different characteristics than the underlying tissue at a wrist. The operation of the biosensor 100 may need to be adjusted in response to its positioning due to such varying characteristics of the underlying tissue. The PPG circuit 110 may adjust a power of the LEDs or a frequency or wavelength of the LEDs based on the underlying tissue. The biosensor 100 may adjust processing of the data. For example, an absorption coefficient may be adjusted when determining a concentration level of a substance based on Beer-Lambert principles due to the characteristics of the underlying tissue.

In addition, the calibrations utilized by the biosensor 100 may vary depending on the positioning of the biosensor. For example, the calibration database may include different table or other correlations between R values and concentration level of a substance depending on position of the biosensor. Due to the different density of tissue and vessels, the R value obtained from measurements over an abdominal area may be different than measurements over a wrist or forehead or fingertip. The calibration database may thus include different correlations of the R value and concentration level depending on the underlying tissue. Other adjustments may also be implemented in the biosensor 100 depending on predetermined or measured characteristics of the underlying tissue of the body part.

### Embodiment—Respiration Rate, Heart Rate and Pulse Pressure

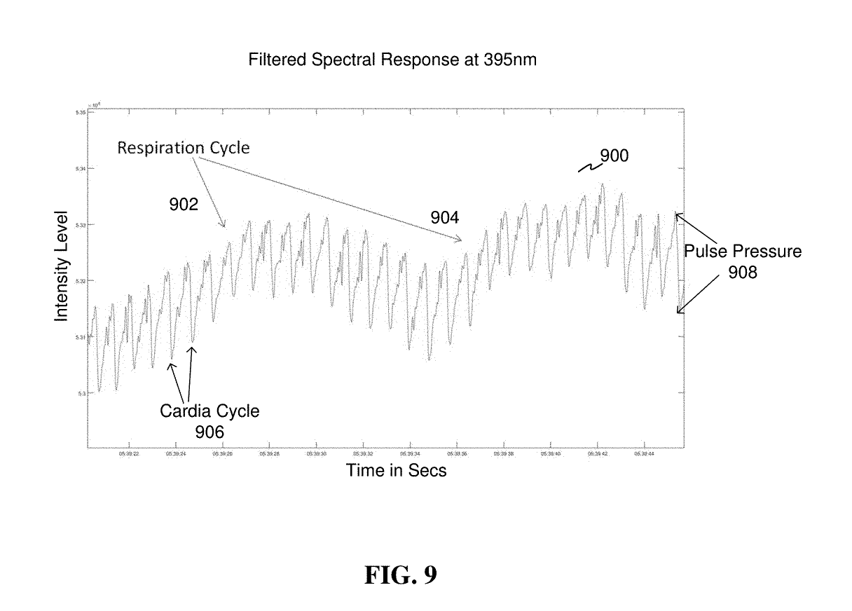

FIG. 9 illustrates a schematic drawing of an exemplary embodiment of a PPG Signal 900 obtained using an embodiment of the biosensor 100 from a user. The PPG Signal 900 was obtained at a wavelength of around 395 nm and is illustrated for a time period of about 40 seconds. The PPG Signal 900 was filtered using digital signal processing techniques to eliminate noise and background interference to obtain the filtered PPG Signal 900. A first respiration cycle 902 and a second respiration cycle 904 may be obtained by measuring a low frequency component or fluctuation of the filtered PPG Signal 900. From this low frequency component, the biosensor 100 may obtain a respiratory rate of a user from the PPG Signal 900.

A heart rate may be determined from the spectral response. For example, the biosensor 100 may determine the time between diastolic points or between systolic points to determine a time period of a cardiac cycle 906. In another embodiment, to estimate the heart rate, the frequency spectrum of the PPG signal is obtained using a FFT algorithm over a predetermined period (hamming window). The pulse rate is estimated as the frequency that corresponds to the highest power in the estimated frequency spectrum. The frequency spectrum may be averaged over a time period, such as a 5-10 second window.

A pulse pressure 908 may be determined from the PPG signal 900. The pulse pressure 908 corresponds to an amplitude of the PPG signal 900 or a peak to peak value. The amplitude of the PPG signal 900 may be averaged over a time period to determine a pulse pressure 908. Thus, a PPG signal may be used to determine heart rate, respiration rate and pulse rate. A light source in the UV range provides a PPG signal with a lower signal to noise ratio for determining heart rate and respiration rate in some tissue while a light source in the IR range provides a PPG signal with a lower signal to noise ratio in other types of tissue. The infrared range (IR) range may include wavelengths from 650 nm to 1350 nm.

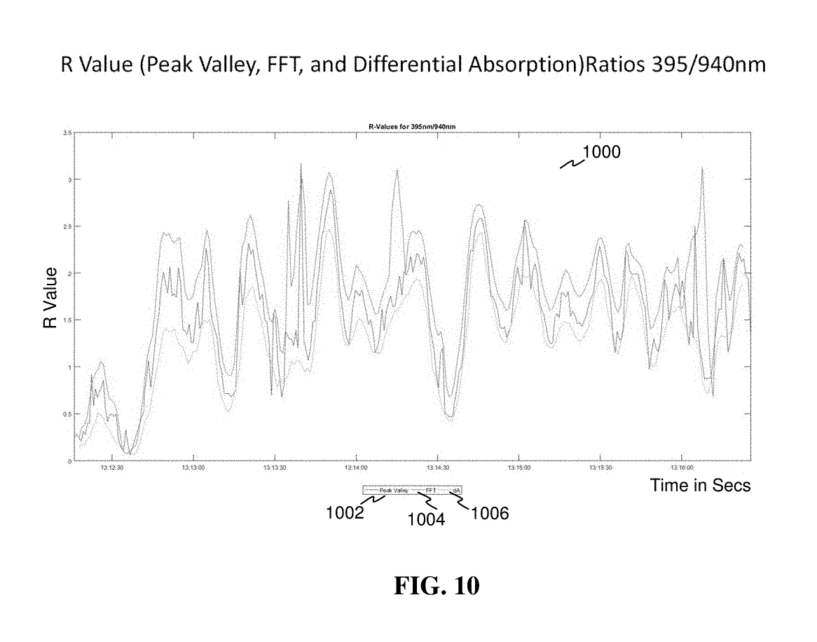

FIG. 10 illustrates a schematic drawing of an exemplary embodiment of results of R values 1000 determined using a plurality of methods. The R values 1000 corresponding to the wavelengths of 395 nm/940 nm is determined using three methods. The R Peak Valley curve 1002 is determined using the Ratio

$$R = \frac{L 395}{L 940}$$

as described hereinabove. The R FFT curve 1004 is obtained using FFT techniques to determine the I_{DC} values and I_{AC} component values of the spectral responses to determine the Ratic

$$R = \frac{L 395}{L 940} .$$

The R differential absorption curve 1006 is determined using the shift in absorbance spectra as described in more detail in U.S. Utility application Ser. No. 15/275,388 entitled, “SYSTEM AND METHOD FOR HEALTH MONITORING USING A NON-INVASIVE, MULTI-BAND BIOSENSOR,” filed Sep. 24, 2016, now U.S. Pat. No. 9,642,578 issued May 9, 2017, and hereby expressly incorporated by reference herein.

As seen in FIG. 10, the determination of the R values using the three methods provides similar results, especially when averaged over a period of time. A mean or average of the R values 1002, 1004 and 1006 may be calculated to obtain a final R value or one of the methods may be preferred depending on the positioning of the biosensor or underlying tissue characteristics.

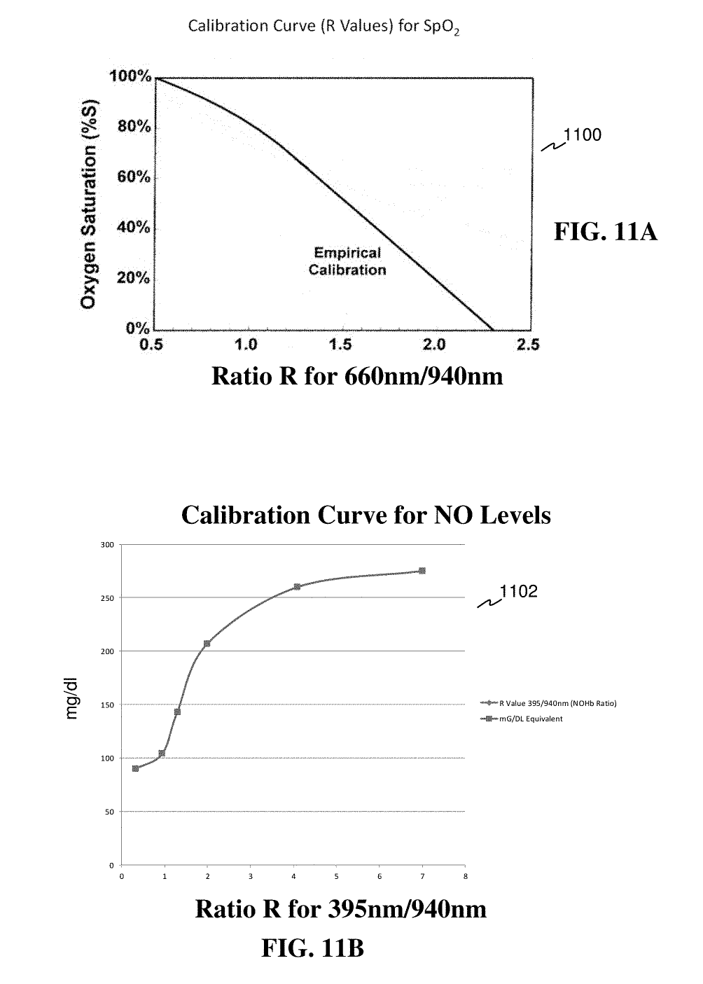

FIG. 11A illustrates a schematic drawing of an exemplary embodiment of an empirical calibration curve 1100 for correlating oxygen saturation levels (SpO₂) with R values. The calibration curve 1100 may be included as part of the calibration database for the biosensor 100. For example, the R values may be obtained for L_{660 nm}/L_{940 nm}. In one embodiment, the biosensor 100 may use a light source in the 660 nm wavelength or in a range of +/−50 nm to determine SpO₂ levels, e.g. rather than a light source in the IR wavelength range. The 660 nm wavelength has been determined in unexpected results to have good results in measuring oxygenated hemoglobin, especially in skin tissue with fatty deposits, such as around the abdominal area.

FIG. 11B illustrates a schematic drawing of an exemplary embodiment of an empirical calibration curve 1102 for correlating NO levels (mg/dl) with R values. The calibration curve 1102 may be included as part of the calibration database for the biosensor 100. For example, the R values may be obtained in clinical trials from measurements of L_{395 nm}/L₉₄₀ nm and the NO levels of a general sample population. The NO levels may be measured using one or more other techniques for verification to generate such a calibration curve 1102. This embodiment of the calibration curve 1102 is based on limited clinical data and is for example only. Additional or alternative calibration curves 1212 may also be derived from measurements of a general population of users at one or more different positions of the biosensor 100. For example, a first calibration curve may be obtained at a forehead, another for an abdominal area, another for a fingertip, another for a palm, etc.

From the clinical trials, the L values obtained at wavelengths around 390 nm (e.g. 380-410) are measuring nitric level (NO) levels in the arterial blood flow. The R value for L390/L940 nm may thus be used to obtain NO levels in the pulsating blood flow. From the clinical trials, it seems that the NO levels are reflected in the R values obtained from L390 nm/L940 nm and wavelengths around 390 nm such as L395 nm/L940 nm. The NO levels may thus be obtained from the R values and a calibration database that correlates the R value with known concentration levels of NO.

In other embodiments, rather than Lλ1=390 nm, the L value may be measured at wavelengths in a range from 410 nm to 380 nm, e.g., as seen in the graphs wherein Lλ1=395 nm is used to obtain a concentration level of NO. In addition, Lλ2 may be obtained at any wavelength at approximately 660 nm or above. Thus, R obtained at approximately Lλ1=380 nm-400 nm and Lλ2>660 nm may also be obtained to determine concentration levels of NO.

In an embodiment, the concentration level of NO may be correlated to a diabetic risk or to blood glucose levels using a calibration database.

### Embodiment—Detection of a Risk of Sepsis or an Infection based on NO levels

In an embodiment, the biosensor 100 may detect a risk of sepsis using NO concentration levels. In this embodiment, an R value derived from L395 and L940 is used to determine an NO measurement though other thresholds may be obtained using other NO measurements, such as R390/940 or L390. In the clinical trials herein, the R395/940 value for a person without a sepsis condition was in a range of 0.1-8. In addition, it was determined that the R395/940 value of 30 or higher is indicative of a patient with a sepsis condition and that the R395/940 value of 8-30 was indicative of a risk of sepsis in the patient. In general, the R395/940 value of 2-3 times a baseline of the R395/940 value was indicative of a risk of sepsis in the patient. These ranges are based on preliminary clinical data and may vary. In addition, a position of the biosensor, pre-existing conditions of a patient or other factors may alter the numerical values of the ranges of the R395/940 values described herein.

The R values are determined by using a wavelength in the UV range with high absorption coefficient for NO, e.g. in a range of 380 nm-410 nm. These R values have a large dynamic range from 0.1 to 300 and above. The percentage variance of R values in these measurements is from 0% to over 3,000%. The R values obtained by the biosensor 100 are thus more sensitive and may provide an earlier detection of septic conditions than blood tests for serum lactate or measurements based on MetHb.

For example, an optical measurement of MetHb in blood vessels is in a range of 0.8-2. This range has a difference of 1.1 to 1.2 between a normal value and a value indicating a septic risk. So, these measurements based on MetHb have less than a 1% percentage variance. In addition, during a septic condition, MetHb may become saturated due to the large amount of NO in the blood vessels. So, an optical measurement of MetHb alone or other hemoglobin species alone is not able to measure these excess saturated NO levels. The R values determined by measuring NO level directly using a wavelength in the UV range are thus more sensitive, accurate, have a greater dynamic range and variance, and provide an earlier detection of septic conditions.

A baseline NO measurement in blood vessels of a healthy general population is obtained. For example, the biosensor 100 may obtain R values or other NO measurements using the biosensor 100. For example, the biosensor 100 may measure an L395 value or determine SpNO% based on an R value for a general population over a period of time, such as hours or days. These NO measurements are then averaged to determine a baseline NO measurement. The NO measurement in blood vessels is then obtained for a general population with a diagnosis of sepsis. For example, the biosensor 100 may obtain R values or other NO measurements (such as an L395 value or SpNO%) for patients diagnosed with sepsis using traditional blood tests, such as serum lactate blood tests. The biosensor 100 may monitor the patients throughout the diagnosis and treatment stages. The NO measurements are then averaged to determine a range of values that indicate a septic condition.

Predetermined thresholds may then be obtained from the NO measurements. For example, a threshold value indicative of a non-septic condition may be obtained. A threshold value for a septic condition may also be obtained. The biosensor 100 is then configured with the predetermined thresholds for the NO measurement.

The predetermined thresholds may be adjusted based on an individual patient's pre-existing conditions. For example, a patient with diabetes may have lower R values. A baseline NO value for a patient may also be determined based on monitoring of the patient during periods without infections. The predetermined thresholds stored in the bio sensor 100 may then be adjusted based on any individual monitoring and/or pre-existing conditions.

In addition, the predetermined thresholds may be determined and adjusted based on positioning of the biosensor 100. For example, different R values or other NO measurements may be obtained depending on the characteristics of the underlying tissue, such as tissue with high fatty deposits or with dense arterial blood flow. The thresholds and other configurations of the biosensor 100 may thus be adjusted depending on the underlying skin tissue, such as a forehead, chest, arm, leg, finger, abdomen, etc.

### Embodiment—Detection of Other Conditions based on NO levels

In another embodiment, post-traumatic stress disorder (PTSD) may result in higher than normal NO levels. There are several reports that increased oxidative stress may be a factor in the evolution of some enduring neurological and psychiatric disorders and PTSD (Bremner, 2006). Stress, a risk factor for developing PTSD, evokes a sustained increase in nitric oxide synthase (NOS) activity that can generate excessive amounts of nitric oxide (Harvey et al., 2004). Oxidation of nitric oxide produces peroxynitrite that is very toxic to nerve cells (Ebadi et al., 2001), and elevated levels of peroxynitrite and its precursor nitric oxide have been observed in patients with PTSD. (Tezcan et al., 2003). The article by Kedar N. Prasad and Stephen C. Bondy, entitled, “Common biochemical defects linkage between post-traumatic stress disorders, mild traumatic brain injury (TBI) and penetrating TBI,” Brain Research, Volume 1599, Pages 103-114, Mar. 2, 2015, and incorporated by reference herein, describes the elevation of nitric oxide NO that may indicate PTSD. The biosensor 100 may operate in one or more modes to detect or provide a warning of abnormal NO levels that may indicate PTSD.

In another embodiment, concussions, mild traumatic brain injury (TBI) and penetrating TBI, may also result in abnormal NO levels. The article by James H. Silver, entitled, “Inorganic Nitrite as a Potential Therapy or Biomarker for Concussion,” J.Neurol Neurophysiol, Volume 7, Issue 2 (April 2016), and incorporated by reference herein, describes an abnormal pattern of nitric oxide NO levels after a concussion. For example, it has been observed that a rapid increase in nitric oxide occurs within minutes following head injury, followed by a decline to below baseline within hours. The biosensor 100 may monitor NO levels after a head trauma and detect this sudden increase and then reduction below baseline in NO levels. In use, a baseline level of NO may be determined for a user during normal conditions. After a potential head injury, the user is then monitored by the biosensor 100 for changes from this baseline level of NO. This process may be performed, e.g., for sideline evaluation of potentially concussed athletes. Thus, the biosensor 100 may operate in one or more modes to monitor NO levels and provide a warning of abnormal NO levels that may indicate a concussion or TBI.

In one or more modes of operation, the biosensor 100 may thus be configured to detect one or more of these other substances in addition to or alternatively from NO levels in blood flow.

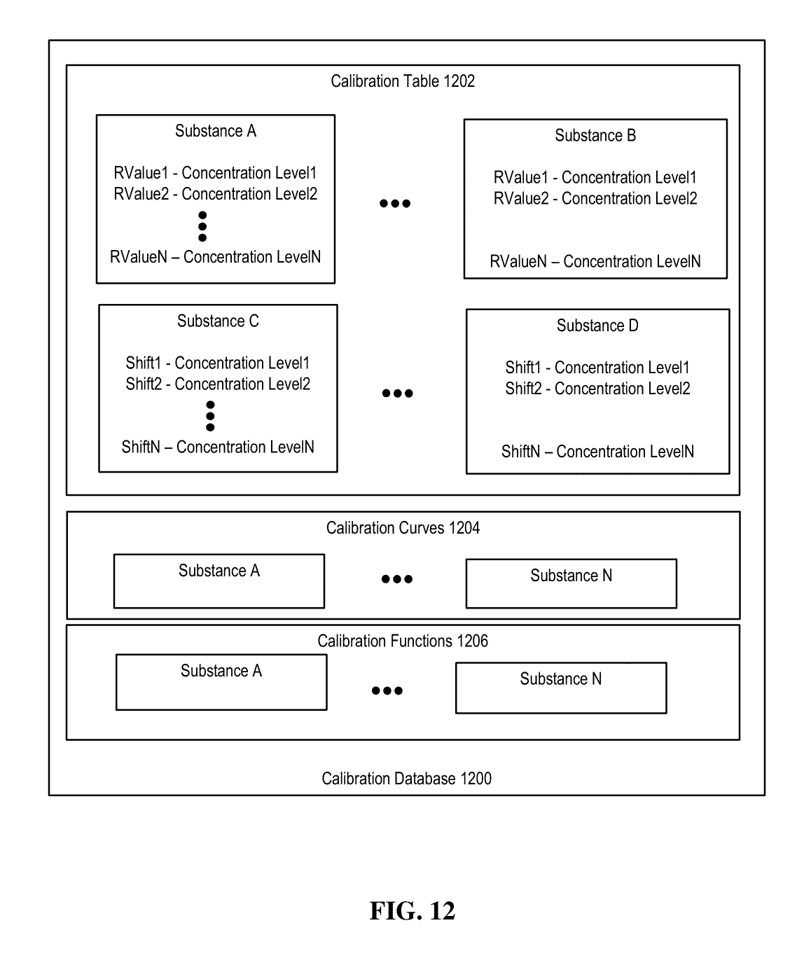

FIG. 12 illustrates a schematic block diagram of an embodiment of a calibration database 1200. The calibration database 1200 includes one or more calibration tables 1202, calibration curves 1204 or calibration functions 1206 for correlating obtained values to concentration levels of one or more substances A-N. The concentration level of the substances may be expressed in the calibration tables 1202 as units of mmol/liter, as a saturation level percentage (SpNO%), as a relative level on a scale (e.g., 0-10), etc.

The calibration database 1200 may also include one or more calibration tables for one or more underlying skin tissue types. In one aspect, the calibration database 1200 may correlate an R value to a concentration level of a substance for a plurality of underlying skin tissue types.

In another aspect, a set of calibration tables 1202 may correlate an absorption spectra shift to a concentration level of one or more substances A-N. For example, a first table may correlate a degree of absorption spectra shift of oxygenated hemoglobin to NO concentration levels. The degree of shift may be for the peak of the absorbance spectra curve of oxygenated hemoglobin from around 421 nm. In another example, the set of calibration tables 1202 may correlate a degree of absorption spectra shift of deoxygenated hemoglobin to NO concentration levels. The degree of shift may be for the peak of the absorbance spectra curve of deoxygenated hemoglobin from around 430 nm.

The calibration database 1200 may also include a set of calibration curves 1204 for a plurality of substances A-N. The calibration curves may correlate L values or R values or degree of shifts of spectral data to concentration levels of the substances A-N.

The calibration database 1200 may also include calibration functions 1206. The calibration functions 1206 may be derived (e.g., using regressive functions) from the correlation data from the calibration curves 1204 or the calibration tables 1202. The calibration functions 1206 may correlate L values or R values or degree of shifts in spectral data to concentration levels of the substances A-N for one or more underlying skin tissue types.

In addition to or alternatively, one or more types of artificial neural networks (a.k.a. machine learning algorithms) may be implemented herein to determine health data as described herein from PPG signals. For example, neural networks may be used to obtain a concentration level of a substance in blood flow, detect an infection, identify type of infection, or other health data using input data derived from PPG signals. Neural network models can be viewed as simple mathematical models defining a function f wherein f:X→Y or a distribution over X or both X and Y. Types of neural network engines or APIs currently available include, e.g. TensorFlow™, Keras™, Microsoft® CNTK™, Caffe™, Theano™ and Lasagne™. The function f may be a definition of a class of functions (where members of the class are obtained by varying parameters, connection weights, thresholds, etc.).

The neural network learns by adjusting its parameters, weights and thresholds iteratively to yield desired output. The training is performed using defined set of rules also known as the learning algorithm. Machine learning techniques include ridge linear regression, a multilayer perceptron neural network, support vector machines and random forests. For example, a gradient descent training algorithm is used in case of supervised training model. In case, the actual output is different from target output, the difference or error is determined. The gradient descent algorithm changes the weights of the network in such a manner to minimize this error. Other learning algorithms include back propagation, least mean square (LMS) algorithm, etc. A set of examples or a training set is used for learning by the neural network. The training set is used to identify the parameters [e.g., weights] of the network.

### Embodiment of Biosensor in Patch Form Factor

The biosensor 100 may be implemented in a patch form factor 1302 that is applied near a wound, surgical incision or other tissue at risk for infection. The patch is preferably positioned near the site of surgery or surgical incisions or a wound. For example, the patch may be placed above the knee prior to a knee replacement surgery.

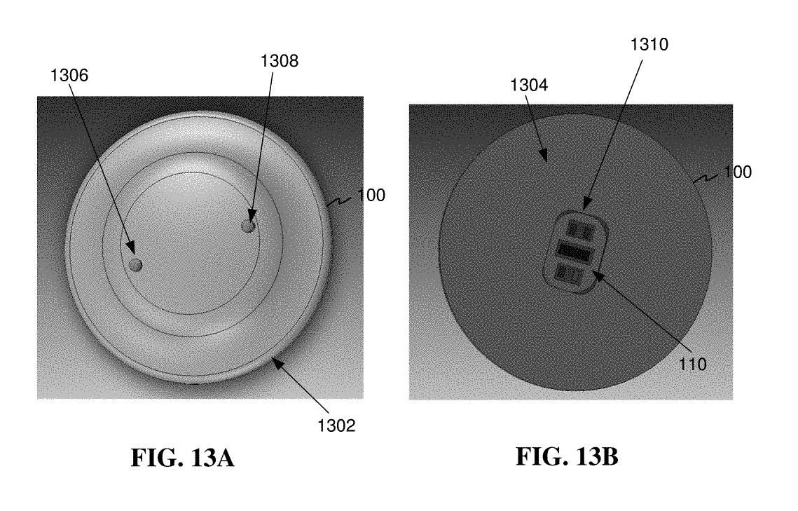

FIG. 13A and FIG. 13B illustrate a perspective view of an embodiment of the biosensor 100 in a patch form factor. FIG. 13A illustrates a perspective front view of the biosensor 100 while FIG. 13B illustrates a perspective back view of the biosensor 100. In this embodiment, the biosensor 100 is included in a disposable patch form factor 1302. The patch 1302 may include an adhesive backing 1304 such that it may adhere to a patient's skin. The patch 1302 may alternatively be secured through other means, such as tape, etc.

The patch 1302 includes an optical sensor or photoplethysmography (PPG) circuit 110. The PPG circuit 110 is configured to emit light through an opening 1310 formed in the adhesive backing 1304. The biosensor 100 further includes one or more health alert indicators 1306, 1308 to provide a warning of possible risk of infection. The health alert indicator 1306 in this embodiment includes a first LED. When symptoms of an infection are detected, the health alert indicator 1306 may illuminate to provide a warning. For example, the health alert indicator 1306 may illuminate a first color (e.g. green) to indicate no or little risk of infection has been detected while a second color (e.g. red) may indicate that symptoms have been detected indicating a risk of sepsis or staph. The biosensor 100 may also measure other patient vitals such as pulse or heart rate, e.g. beats per minute (bpm), respiration rate and temperature that are indicated by a second health alert indicator 1308. The first and second indicators 1306, 1308 may alternatively include audible or digital displays.

Due to its compact form factor, the patch 1302 may be attached on various skin surfaces of a patient, including on a forehead, arm, wrist, abdominal area, chest, leg, hand, etc. The patch 1302 in an embodiment may be designed to be disposable, e.g. designed to be used on a single patient. For example, the biosensor 100 may include a battery with a relatively short life span of 24-48 hours.

The biosensor 100 records biosensor data of the patient at a wound site or during surgery and thereafter during recovering. The biosensor 100 may wirelessly transmit biosensor data to another device, such as a monitor or mobile device. The biosensor 100 may thus provide localized data for monitoring a wound during surgery, post-surgery and recovery. The biosensor 100 may detect any changes indicating an infection, identify a type of infection and store patient vitals and health information for the patient.

Embodiment of Detection of Sepsis

The biosensor 100 may identify a risk of infection or sepsis. For example, the biosensor 100 obtains an NO concentration level from an R value using L_{λ1}=390 nm and L_{λ2}=940 nm or an R value at L_{λ1}=395 nm and L_{λ2}=660 nm. In another embodiment, the NO measurement may be obtained using a value of L₁=380 nm-400 nm and L_{λ2}≥660 nm. The spectral response used to determine the value of L₁=380nm-400 nm may also be measuring other NO compounds or isoforms such as eNOS or iNOS or nNOS or other compounds bonded to a plurality of hemoglobin species. The concentration of the plurality of hemoglobin species may be adjusted from the NO measurements and a calibration database to obtain an NO concentration level. In another example, the biosensor 100 may determine the relative shift in the absorbance spectra for a substance (such as hemoglobin) and access a calibration database that correlates the relative shift in the absorbance spectra of the substance with a concentration level of NO.

The biosensor 100 may display the baseline NO measurement and then non-invasively and continuously monitor the NO measurement in blood vessels. For example, the biosensor 100 may obtain the NO measurement at least once per minute or more frequently, such as every 10 seconds or 30 seconds, and continues to display the NO measurement. The biosensor 100 may also monitor other patient vitals indicative of sepsis condition, such as temperature, pulse, and respiration rate.

The NO measurement is then compared to a first predetermined threshold. For example, normal ranges of the NO measurement from the baseline measurement are determined for septic risk. Patient vitals may also be compared to predetermined thresholds. Depending on the comparison, one or more warnings are displayed. For example, the first predetermined threshold may be when the NO measurement has exceeded at least 10% of the baseline level of the NO measurement. A warning is displayed to indicate a health alert. A caregiver may then perform other tests to determine the cause of the elevated NO measurement, such as lactic acid blood test for sepsis.

The biosensor continues to monitor the NO measurement in blood vessels and compare the NO measurement to one or more predetermined thresholds. In 1812, it is determined that the NO measurement has exceeded a second predetermined threshold. For example, the NO measurement equals or exceeds at least 30% of a baseline level of the NO measurement. A warning to indicate a medical emergency is displayed at 1814. Due to the immediate danger of such high levels of NO measurement and dangers of septic shock, a request for immediate emergency treatment may be indicated. Though 10% and 30% are illustrated in this example, other percentages over the baseline level may also trigger warnings or alerts.

TABLE 1
SpNO % Interpretation (NO Levels)

| | |
|---|---|
| 0-1.5% | Diabetic Patients |
| 1.5-2% | Pre-Diabetic |
| 2-8% | Normal |
| >10% | Clinically significant, consult medical caregiver for direction |
| >30% | Assess for septic shock, provide high flow O₂, and transport |

### Embodiment—Adjustments in Response to Positioning of the Biosensor

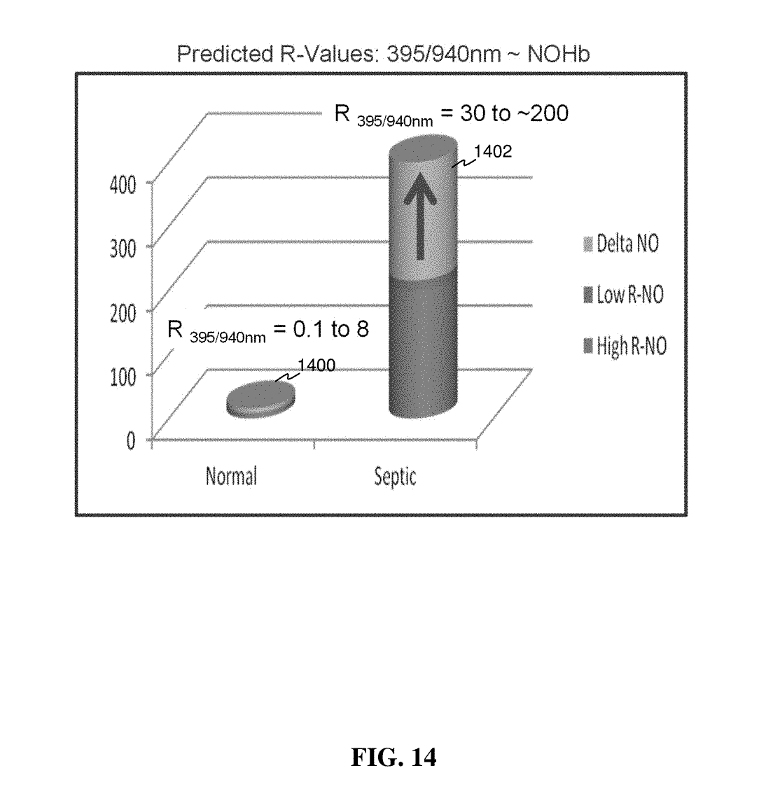

FIG. 14 illustrates a schematic block diagram of an embodiment of predetermined thresholds of NO measurements for detecting a risk of sepsis. In this embodiment, an R value using L₃₉₅ and L₉₄₀ is illustrated as the NO measurement though other thresholds may be obtained using other NO measurements, such as R_{390/940} or L₃₉₀. In the clinical trials herein, the R_{395/940} value for a person without a sepsis condition was in a range of 0.1-8. In addition, it was determined that an R value of 30 or higher is indicative of a patient with a sepsis condition and that an R value of 8-30 was indicative of a risk of sepsis in the patient. In general, an R value of 2-3 times a baseline R value was indicative of a risk of sepsis in the patient.

For example, in the example shown in FIG. 14, a range 1400 of the R value is from 0.1 to 8 for a person without a sepsis condition. The range 1402 of the R value for a person with a sepsis risk is from 30 to 200 or above. These ranges are based on preliminary clinical data and may vary. In addition, a position of the biosensor, pre-existing conditions of the patient or other factors may alter the numerical values of the ranges of the R values described herein.

The R values are determined by measuring NO concentration level directly using a wavelength in the UV range with high absorption coefficient for NO, e.g. in a range of 380 nm-410 nm. These R values have a large dynamic range from 0.1 to 300 and above. The percentage variance of R values in these measurements is from 0% to over 3,000%. The R values obtained by the biosensor 100 are thus more sensitive and may provide an earlier detection of septic conditions than blood tests for serum lactate or measurements based on MetHb.

For example, an optical measurement of MetHb in blood vessels is in a range of 0.8-2. This range has a difference of 1.1 to 1.2 between a normal value and a value indicating a septic risk. So, these measurements based on MetHb have less than a 1% percentage variance. In addition, during a septic condition, MetHb may become saturated due to the large amount of NO in the blood vessels. So, an optical measurement of MetHb alone or other hemoglobin species alone is not able to measure these excess saturated NO levels. The R values determined by measuring NO level directly using a wavelength in the UV range are thus more sensitive, accurate, have a greater dynamic range and variance, and provide an earlier detection of septic conditions.

In an embodiment, the biosensor 100 may be configured with corresponding thresholds to trigger one or more health alerts. For example, the biosensor 100 may be configured to indicate a non-septic range of NO levels for R₃₉₅₁₉₄₀ values from 0.1 to 8. For R_{395/940} values from 8 to 30, the biosensor 100 may indicate a risk of sepsis or infection. A healthcare provider may determine to continue monitoring or perform additional tests or begin a treatment for infection. For R₃₉₅₁₉₄₀ values at 30 or above, the patch may be configured to indicate a second alert indicating a high health risk or onset of sepsis. A healthcare provider may determine to immediately begin an aggressive treatment for infection or perform additional treatments and intervention.

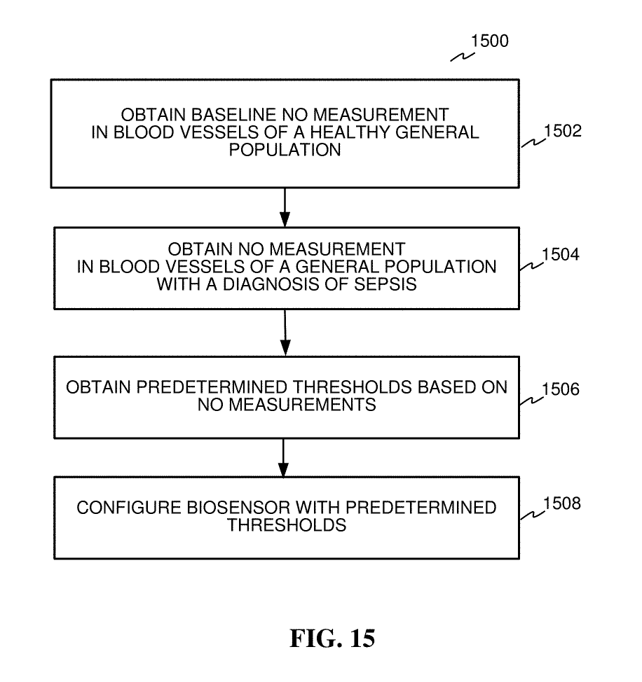

FIG. 15 illustrates a logical flow diagram of an embodiment of a method 1500 for determining predetermined thresholds for health alert indicators for sepsis. A baseline NO measurement in blood vessels of a healthy general population is obtained in 1502. For example, the biosensor 100 may obtain R values or other NO measurements using the biosensor 100. For example, the biosensor 100 may measure an L₃₉₅ value or determine SpNO% based on an R value for a general population over a period of time, such as hours or days. These NO measurements are then averaged to determine a baseline NO measurement.

The NO measurement in blood vessels is then obtained for a general population with a diagnosis of sepsis at 1504. For example, the biosensor 100 may obtain R values or other NO measurements (such as an L₃₉₅ value or SpNO%) for patients diagnosed with sepsis using traditional blood tests, such as serum lactate blood tests. The biosensor 100 may monitor the patients throughout the diagnosis and treatment stages. The NO measurements are then averaged to determine a range of values that indicate a septic condition.

Predetermined thresholds may then be obtained from the NO measurements at 1506. For example, a threshold value indicative of a non-septic condition may be obtained. A threshold value for a septic condition may also be obtained. The biosensor 100 is then configured with the predetermined thresholds for the NO measurement at 1508.

The predetermined thresholds may be adjusted based on an individual patient's pre-existing conditions. For example, a patient with diabetes may have lower R values. A baseline NO value for a patient may also be determined based on monitoring of the patient during periods without infections. The predetermined thresholds stored in the bio sensor 100 may then be adjusted based on any individual monitoring and/or pre-existing conditions.

A similar process may also be performed for other parameters in the detection of sepsis and identifying a type of sepsis. For example, a baseline measurement in a healthy population may be obtained of the color of the blood flow, level of liver enzymes, vasodilation, heart rate, respiration rate or skin temperature. The parameters may then be measured in patients diagnosed with febrile and afebrile sepsis and threshold values for a septic condition may be obtained for one or more of the other parameters.

In addition, the predetermined thresholds may be determined and adjusted based on positioning of the biosensor 100. For example, different R values or other NO measurements may be obtained depending on the characteristics of the underlying tissue, such as tissue with high fatty deposits or with dense arterial blood flow. The thresholds and other configurations of the biosensor 100 may thus be adjusted depending on the underlying skin tissue, such as a forehead, chest, arm, leg, finger, abdomen, etc.

When the biosensor 100 is implemented in the patch 1302 form factor, the biosensor 100 may be positioned over different areas of a patient. The skin tissue exhibits different underlying characteristics depending on the area of the body. For example, the biosensor 100 may be positioned on or attached to, e.g. a hand, a wrist, an arm, forehead, chest, abdominal area, ear lobe, fingertip or other area of the skin or body or living tissue. The characteristics of underlying tissue vary depending on the area of the body, e.g. the underlying tissue of an abdominal area has different characteristics than the underlying tissue at a wrist. The operation of the biosensor 100 may need to be adjusted in response to its positioning due to such varying characteristics of the underlying tissue.

The biosensor 100 then correlates the detected characteristics of the underlying tissue with known or predetermined characteristics of underlying tissue (e.g. measured from an abdominal area, wrist, forearm, leg, forehead, etc.) to determine its positioning. Information of amount and types of movement from an activity monitoring circuit implemented within the biosensor 100 may also be used in the determination of position. The PPG circuit 110 may adjust a power of the LEDs or a frequency or wavelength of the LEDs based on the underlying tissue. The biosensor 100 may adjust processing of the data at 1908. For example, an absorption coefficient may be adjusted when determining a concentration level of a substance based on Beer-Lambert principles due to the characteristics of the underlying tissue. In addition, the calibrations utilized by the biosensor 100 may vary depending on the positioning of the biosensor. For example, the calibration database may include different table or other correlations between R values and NO concentration level depending on position of the biosensor. Due to the different density of tissue and vessels, the R value obtained from measurements over an abdominal area may be different than measurements over a wrist or forehead. The calibration database may thus include different correlations of the R value and NO concentration level depending on the underlying tissue. Other adjustments may also be implemented by the biosensor 100 depending on predetermined or measured characteristics of the underlying tissue. The biosensor 100 is thus configured to obtain position information and perform adjustments to its operation in response to the position information.

### Embodiment of Identification of Types of Sepsis Infections

In one or more embodiments herein, the biosensor may identify a type of sepsis infection based on a testing of various parameters. For example, the biosensor 100 may obtain one or more of the following parameters: skin temperature, liver enzyme levels (such as P450 Cytochrome), NO levels, heart rate, vasodilation levels, or respiration rate. Using these parameters, the biosensor 100 identifies a risk of, e.g., Febrile or AFebrile Sepsis.

For example, the AFebrile Sepsis condition generally exhibits extreme skin temperature variations, and the Nitric Oxide levels in the blood (>X level) are likely to vary with elevated spikes as well. The Febrile Sepsis condition generally exhibits a more constantly elevated Nitric Oxide levels (>Y level) without large skin temperature swings.

The biosensor 100 may non-invasively and continuously monitor these parameters for early detection of sepsis and discernment of the type of sepsis condition, such as Febrile or AFebrile Sepsis. Table 2 below illustrates an embodiment of exemplary parameters for testing by the biosensor 100 to determine whether a sepsis infection includes a Febrile or AFebrile type infection.

TABLE 2

| Parameters | Febrile | Afebrile |
|---|---|---|
| Skin Temperature | Low Grade Fever | High Grade Fever |
| Liver Enzyme Level | Elevated | Abnormal Elevation in Waves |
| NO Level | NOHb Rvalue >6 | NOHb Rvalue >8 |
| Vasodilation | Small Range of | Larger Range of Vasodilation |
| | Vasodilation | |
| Heart Rate, | | Heart Rate Elevated >120, |
| Respiration | | Respiration Rate is High |

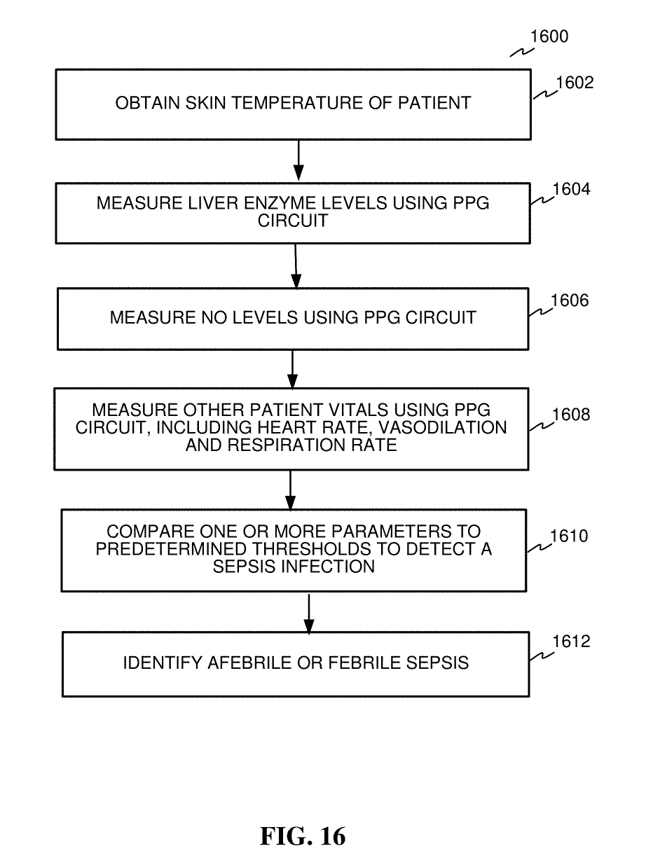

FIG. 16 illustrates a logical flow diagram of an exemplary embodiment of a method 1600 for identifying Febrile or AFebrile Sepsis by the biosensor 100. The biosensor 100 non-invasively obtains a skin temperature of a patient, at periodic intervals, e.g. one or more times per minute at 1602.

The biosensor 100 non-invasively obtains a concentration level of one or more liver enzymes in blood flow of a patient, at periodic intervals, e.g. one or more times per minute at 1604. For example, the biosensor 100 may be configured to obtain an indicator of a concentration level of a liver enzyme, such as P450 or NAD+ or NADH or another ADH related enzyme. In unexpected results, the R_{468,940} ratio measures the liver enzyme called cytochrome P450 Oxidase (P450). In one aspect, a spectral response around a wavelength at approximately 468 nm was used by the biosensor 100 to obtain L values that tracked the concentration levels of P450. Thus, the measurement of the spectral response for the wavelength at approximately 468 nm or in ranges around 468 nm (such as +/−15 nm) to indicate concentration levels of the liver enzyme P450. A calibration table then includes a graph, table, or other means to correlate P450 concentration levels with values for the R_{468/940} ratio.

The biosensor 100 may also be configured to detect an indicator of a concentration level of other liver enzymes, such as ADH related enzyme NADH. It has been found that the molecule NADH has an absorbance coefficient that is greatest around approximately 340 nm while the molecule NAD+ has an absorbance coefficient that is low around 340 nm. The PPG circuit 110 may obtain a plurality of spectral responses from light reflected or transmitted through skin around 340 nm or in ranges around 340 nm (such as +/−15 nm) and obtain a ratio R=L_{340 nm}/L_{940 nm}. Though 940 nm is described herein, other wavelengths may be utilized with a low absorbance coefficient for NADH. The biosensor 100 obtains values for the R_{340/940} ratio as an indicator of a concentration level of NADH. The values for the R_{340/940} ratio may then be correlated using a sample general population, to generate a calibration table. The calibration table would include a graph, table, or other means to correlate concentration NADH levels with values for the R_{340/940} ratio. Though measurement of a level of NADH and P450 is described herein, the biosensor 100 may be configured to measure other liver enzymes.

The biosensor also monitors concentration levels of nitric oxide (NO) in the blood stream using the PPG circuit at 1606. The biosensor 100 non-invasively obtains the NO levels in arterial blood flow of a patient, at periodic intervals, e.g. one or more times per minute. In general, it has been determined from initial clinical trials that the average R value may range from 0.1 to 8 for a patient without a sepsis condition. In addition, it was determined that an average R value of 30 or higher is indicative of a patient with a sepsis condition and that an average R value of 8-30 was indicative of a risk of sepsis in the patient. In general, an R value of 2-3 times a baseline R value was indicative of a risk of sepsis in the patient.

The biosensor may also measure other patient vitals using the PPG circuit, such as heart rate, respiration rate and vasodilation at 1608. The one or more parameters are compared to predetermined thresholds to detect a sepsis infection is present at 1610. In addition, the type of sepsis infection, such as Afebrile or Febrile sepsis is identified at 1612. For example, the AFebrile Sepsis condition generally exhibits extreme skin temperature variations under care (waves and/or spikes) and the Nitric Oxide levels in the blood (>X threshold level) are more likely to spike similarly. The Febrile sepsis condition generally exhibits a more constantly high Nitric Oxide level (>Y threshold level) without large skin temperature swings. Using one or more of these parameters, the biosensor 100 identifies the type of sepsis.

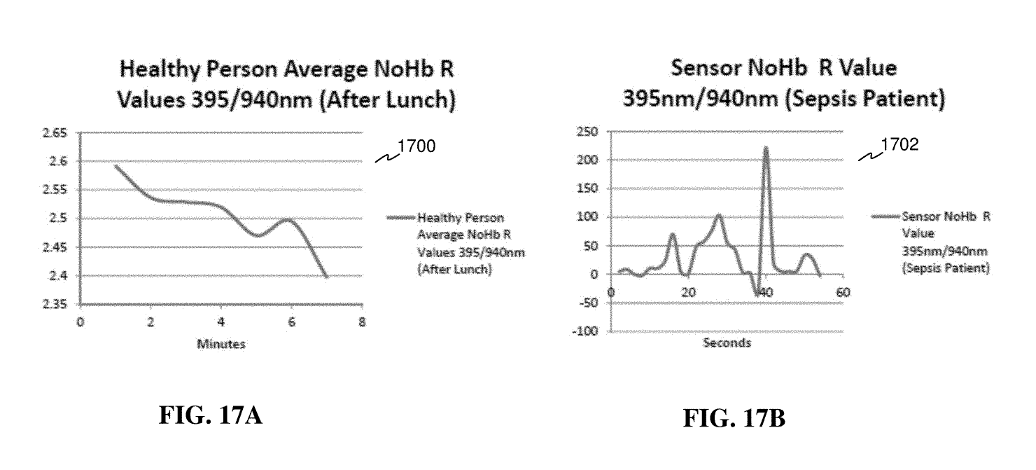

FIG. 17A illustrates a graphical diagram 1700 of an embodiment of clinical data for NO measurements of a healthy person. The biosensor 100 measured the R values at R_{395 nm/940 nm} of a healthy patient. As seen in the graphical diagram 1700, the R values range from around 2.6 to 2.4 over a period of about six minutes.

FIG. 17B illustrates a graphical diagram 1702 of an embodiment of clinical data for NO measurements of a patient with sepsis. The patient was diagnosed with sepsis using one or more currently known blood tests. The biosensor 100 measured the R values at R_{395 nm/940 nm} of the patient. As seen in the graphical diagram 1702, the R values spike over the measurement period and fluctuate to over 200 during the measurement period of about one minute. The NO measurements spike up 6 to 10 times higher in the sepsis patient than a healthy patient. Due to the fluctuation and spikes of the NO measurements, this symptom indicates Afebrile Sepsis. The biosensor 100 may thus detect a sepsis infection and identify an AFebrile type sepsis infection.

### Embodiment of Detection of Hemolysis

Hemolysis or haemolysis, also known by several other names, is the rupturing (lysis) of red blood cells (erythrocytes) and the release of their contents (cytoplasm) into surrounding fluid (e.g. blood plasma). Hemolysins damage the host cytoplasmic membrane, causing cell lysis and death. Hemolysis may be caused by various medical conditions, including many Gram-positive bacteria (e.g., Streptococcus, Enterococcus, and Staphylococcus), some parasites (e.g., Plasmodium), some autoimmune disorders (e.g., drug-induced hemolytic anemia), some genetic disorders (e.g., Sickle-cell disease or G6PD deficiency), or blood with too low of a solute concentration (cells in hypotonic solution).

Currently, hemolysis is observed and measured with in vitro assays involving the lysis of red blood cells (erythrocytes). Problems exist with in vitro assays since hemolysis can be caused by improper techniques during collection of blood specimens, by the effects of mechanical processing of blood, or by bacterial action in cultured blood specimens.

In an embodiment, the biosensor 100 may detect a presence of hemolysis in blood flow. If as little as 0.5% of the red blood cells are hemolyzed, the released hemoglobin will cause the serum or plasma to appear pale red or cherry red in color. This change in color may be measured in vivo by the biosensor 100. The biosensor 100 may detect an abnormal color of the blood flow in vivo and determine a percentage of RBCs affected. For example, the PPG circuit 110 may detect a change in color of the arterial pulsatile blood flow due to hemolysis and correlate the change in color to a percentage of affected red blood cells.

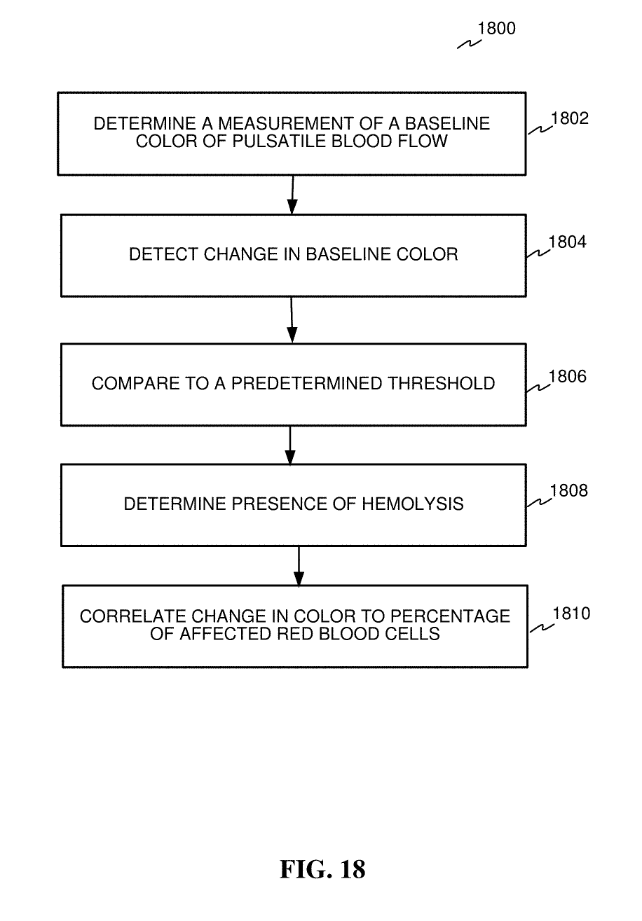

FIG. 18 illustrates a logical flow diagram of a method 1800 for detection of hemolysis. In an embodiment, the biosensor 100 detects a baseline color of pulsatile blood flow at 1802. The baseline color may be an average or mean color hue of the individual or an average or mean color or hue of an average healthy population, preferably with a same blood type. The biosensor 100 may then monitor the color of the blood flow to detect changes in 1804. The biosensor 100 may use one or more wavelengths in visible range and determine an intensity of the reflected one or more visible wavelengths (e.g., Red, Yellow, or Green wavelengths). The color or change in color is then compared to a predetermined threshold at 1806. The predetermined threshold is based on testing of color of blood samples with known hemolysis. For example, when a percentage 0.5% of the red blood cells are hemolyzed, the released hemoglobin will cause the serum or plasma to exhibit a pale red or cherry red color.

When the biosensor 100 determines the change exceeds the predetermined threshold, the biosensor 100 determines a presence of hemolysis at 1808. The biosensor 100 may correlate the change in color of the blood flow to a level of affected red blood cells (such as percentage or count of RBCs), e.g. using a calibration table, algorithm or curve at 1810. The biosensor 100 may thus detect a presence of hemolysis in blood flow and correlate the change in color to a level of affected red blood cells.

### Embodiment of Identification of Types of Staph Infections

In one or more embodiments herein, the biosensor 100 includes an early warning system and method for detection of a risk of a staph infection. In addition, the biosensor 100 may identify a type of staph infection based on a testing of various parameters. For example, the biosensor 100 monitors one or more of the following parameters: Red Blood Cells (RBC), skin temperature, liver enzyme levels (P450, NAHD, and/or NAD+), NO levels, vasodilation levels, heart rate, SpO₂, or respiration rate.

The two most common types of staph infections include Hemolytic and Non-Hemolytic varieties. In general, red blood cells in blood flow exhibit abnormal optical properties in Hemolytic type staph infections. Additionally, the liver enzyme levels (P450) exhibit peaks and waves in Non-Hemolytic staph infections. The Nitric Oxide levels also exhibit higher levels in Non-Hemolytic staph infections.

Table 3 below illustrates an embodiment of exemplary parameters for testing by the biosensor 100 to identify a type of infection as a Hemolytic or Non-Hemolytic type staph infection.

TABLE 3

| Parameters | Hemolytic | Non-Hemolytic |
|---|---|---|
| Color of Blood Flow | Abnormal | Normal |
| Skin Temperature | Low Grade | High Temperature in Waves |
| Liver Enzyme Level | Abnormal | Abnormal High in Waves |
| NO Level | NOHb Rvalue >7 | NOHb Rvalue >15 in peaks |
| Vasodilation | Small Range of | Larger Range of |
| | Vasodilation | Vasodilation |

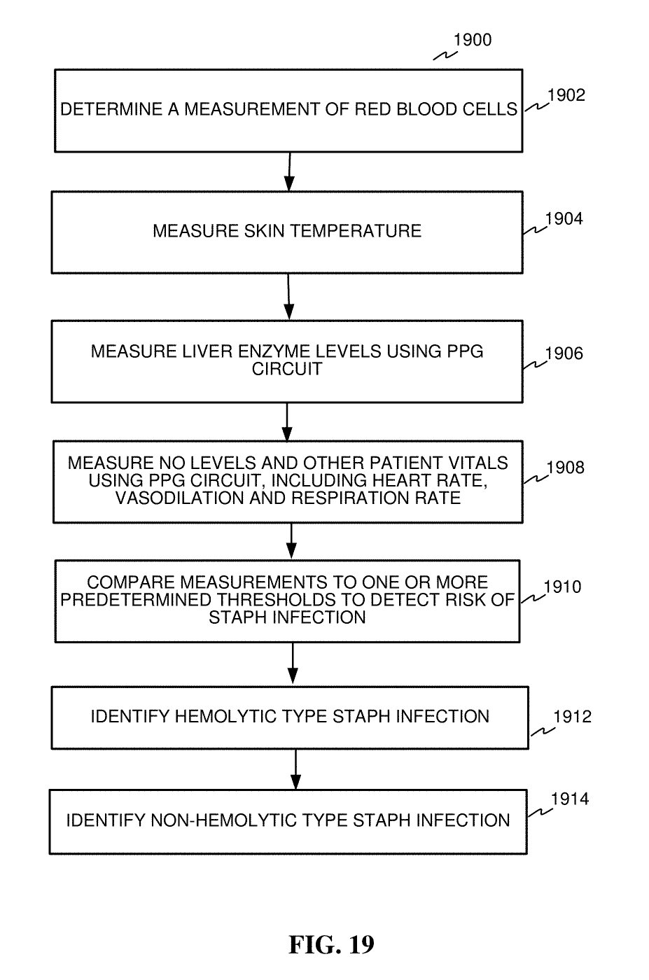

FIG. 19 illustrates a logical flow diagram of a method 1900 for detecting a staph infection and identifying a type of staph infection, and in particular, a Hemolytic and Non-Hemolytic staph infection by the biosensor 100. The PPG circuit 110 of the biosensor 100 is configured to determine a measurement of red blood cells, such as a color of the blood flow at 1902. The biosensor 100 may also measure a count of the red blood cells using an antigen marker detection as described in more detail below.

The biosensor 100 also non-invasively obtains a skin temperature of a patient, at periodic intervals, e.g. one or more times per minute at 1904. The biosensor may also monitor liver enzyme levels in the blood stream using the PPG circuit at 1906. The biosensor 100 non-invasively obtains the liver enzyme levels in blood flow of a patient, at periodic intervals, e.g. one or more times per minute. The PPG circuit may also measure NO levels in blood flow of a patient, at periodic intervals, e.g. one or more times per minute at 1908. The PPG circuit may also determine other patient vitals, such as heart rate, local vasodilation and respiration rate.

The various measured parameters are compared to predetermined thresholds to detect a risk of a staph infection at 1910. In addition, the type of staph infection may be determined, such as Hemolytic and Non-Hemolytic varieties. In an embodiment, the Red Blood Cells exhibit abnormal optical properties in Hemolytic staph infections versus Non-Hemolytic varieties of Staph. For example, the RBC exhibit an abnormal composition since a destruction of RBCs occurs during the infection period. Hemolysis is the rupturing or RBCs and the release of their contents (cytoplasm) into surrounding fluid (e.g. blood plasma). The biosensor 100 may detect hemolysis through an abnormal count of RBCs and/or color of the blood flow.

Additionally, the P450 Levels are expected to peak with waves in Staph Non-Hemolytic infections vs Hemolytic versions (>X Level). In addition, the Nitric Oxide levels are expected to be higher in Non-Hemolytic variety staph infections. These and other parameters may thus be measured by the biosensor 100 to determine a type of staph infection.

The biosensor 100 may thus measure one or more of these parameters and identify a hemolytic type staph infection in 1912 or identify a non-hemolytic type staph infection in 1914. A care giver may thus detect an infection and identify a type of infection to proceed with treatment or further analysis.

### Overview of Use of Biosensor for Detection of Infection

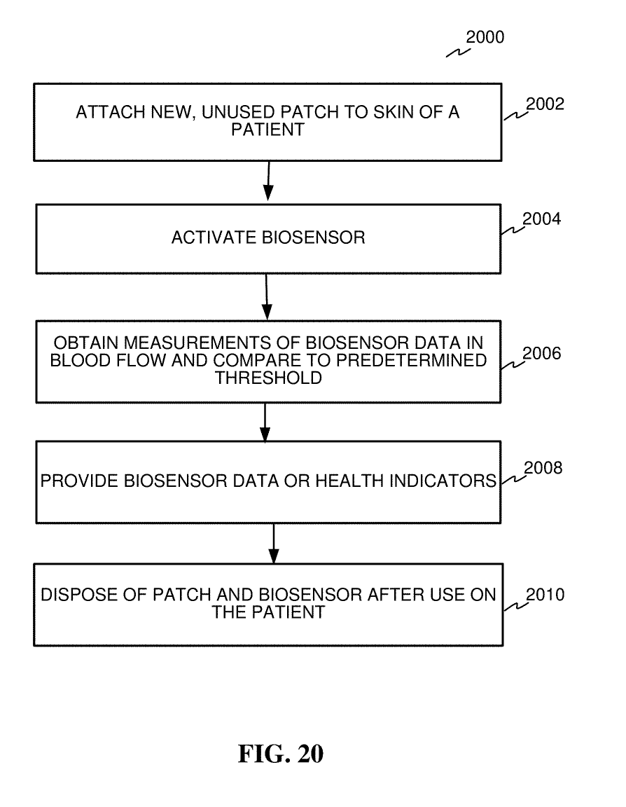

FIG. 20 illustrates a logical flow diagram of a method 2000 for detection of an infection and identification of a type of infection. In an embodiment, the biosensor 100 is integrated in a disposable patch that is applied to skin of a patient at 2002. For example, the patch is applied to a wound upon admittance to a hospital or positioned on or near an incision site during surgery or surgical incisions. The bio sensor 100 may then be activated at 2004. The biosensor 100 continuously and non-invasively measures substances in blood flow as well as patient vitals at 2006. The biosensor 100 may also determine a blood type of a patient as described in more detail below. The biosensor 100 may also monitor levels of red blood cells (RBC) and white blood cells (WBC) as described in more detail below.

The biosensor 100 may record the measurements of biosensor data, such as patient vitals, blood type, RBC or WBC levels and concentration level of one or more substances in blood flow. The biosensor 100 may wirelessly transmit the biosensor data to a monitor or electronic medical record of the patient. When one or more measurements of the biosensor data exceed predetermined thresholds, the biosensor 100 may provide a health alert, display or indicator at 2008. For example, the biosensor 100 may provide an indicator of an infection and/or type of infection. The biosensor 100 may provide an indicator of an increase or high level of white blood cells or hemolysis. The patch and biosensor 100 may be disposed after one use on the single patient at 2010.

The biosensor 100 may thus provide biosensor data for monitoring incisions during surgery and post-surgery follow up. The biosensor may also be applied to wounds or other sites at risk for infection. The biosensor 100 may detect biosensor data indicating a change in white blood cell levels or red bloods cell levels or an infection and transmit the biosensor data to an electronic medical record (EMR) or monitoring device.

### Embodiment of Detection of Red Blood Cells and Blood Type

Blood type is represented by the ABO and Rh(D) systems. The A, B, O, AB blood type of a person depends on the presence or absence of two genes, A and B. These genes determine the configuration of the red blood cell surface. A person who has two A genes has red blood cells of type A. A person who has two B genes has red cells of type B. If the person has one A and one B gene, the red cells are type AB. If the person has neither the A nor the B gene, the red cells are type O. It is essential to match the ABO status of both donor and recipient in blood transfusions and organ transplants. In addition to the four main blood groups—A, B, AB and O, each group can either be RhD positive or RhD negative. Antigens are proteins on the surface of blood cells that can cause a response from the immune system. The Rh factor is a type of protein on the surface of red blood cells. Most people who have the Rh factor are Rh-positive. Those who do not have the Rh factor are Rh-negative. This means that in total there are eight main blood groups.

The optical properties of the different blood groups can be detected. The red blood cells comprise about 45% of the human blood. The color differences between the different blood groups is detectable by the PPG circuit. Thus, the optical differences between the different RBC groups (A,B, AB and O) can be established using the PPG circuit. The spectral differences of the antigens present allow for several methods to be developed to determine the basic blood grouping using the multi-wavelength PPG circuit 110. For example, in the Armenian Journal of Physics, 2011, vol. 4, issue 3 pp. 165-168 shows a method using Blood grouping detection using fiber optics. The basic premise of the method described is to use a laser operating at 820 nm to fire a series of pulses into a blood sample at 10 Khz, then using a photo diode, convert the optical variations back into electrical variations by amplifying, filtering, rectifying and feed the primary signal into a capacitor filter. This capacitor changes a voltage which is different for various blood groups. Since the different blood types have different optical spectrum characteristics, this method of fiber optic injection into a blood sample and then reading the approximate integration response of the corresponding signal shows a basic mathematical integration method is possible. However this method requires a raw blood sample and is expensive and time consuming.

Since the different blood groups have different optical spectrums due to variances in the antigen groups, the PPG circuit 110 described herein may be used to measure the spectral response of blood flow using multiple wavelengths and comparing the R values between them to estimate a particular blood group. The various R values indicate a presence of an antigen to identify a blood group of A, B, O or AB using the plurality of spectral responses. The PPG circuit may use the same R values or different R values to determine a presence of another antigen within a blood group to identify an RH factor using the plurality of spectral responses.

The biosensor 100 described herein may be configured to assess the blood group of a patient using the PPG circuit 110. In an embodiment, the PPG circuit 110 emits a series of pulses at a patient's tissue to obtain a series of R values. The series or average of the series of R values is used to identify a blood type, e.g. the antigen group present or absent on blood cells. The PPG circuit 110 uses a series of pulses firing LED's at a rate of between 100-200 Hz to obtain a good heart rate signal. One or more of the following R values for 550/940 nm, 660/940 nm, and 880 nm/940 nm frequencies may be obtained over an integration of a series of heartbeats. Due to the division of the L values, the R value eliminates the input from the skin tissue and non-pulsating blood flow to isolate the input from the pulsating blood flow (venous or arterial). To determine a blood group, the R values may be obtained over a sample window, such as over a plurality of heartbeats. A blood group indicator may be derived from the values of the R ratio over the sample window. For example, an integration of the R values over the sample window may be determined and then the integrated R values used as the blood group indicator. The blood group indicator is then used to identify a blood group from one or more blood group reference tables.

For example, in order to enhance the data signal of a spectral response, the data signal in a spectral response over a series of heart beats is used for the sample window. The R value may be obtained over the sample window using spectral responses around a plurality of frequencies. The frequencies may include, e.g., 550, 660 and 880 nm frequencies or in ranges of wavelengths around such frequencies. In one embodiment, the frequencies include 530 and 590 nm and values for the ratio R=L_{530/L940} and R=L_{590/L940} are determined over the sample window. The values for the first R_{530/940} ratio are then integrated across the sample window to determine an integrated R value as a first blood group indicator. The values for the second R_{590/940} ratio are then integrated across the sample window to determine an integrated R value as a second blood group indicator. A simple integration algorithm for each individual frequency may be implemented to obtain the blood group indicators. In another embodiment, the values for the R ratios are averaged over the sample window. Other functions using the values of the R ratios over the sample window may be implemented to obtain one or more blood group indicators.

The obtained one or more blood group indicators are then used with a calibration table to identify a blood group of the patient, human or animal. For example, the calibration table includes a correlation of values or ranges of the one or more blood group indicators to blood group or blood type. The calibration table may be determined by obtaining the blood group indicator for a sample general population for each known blood type.

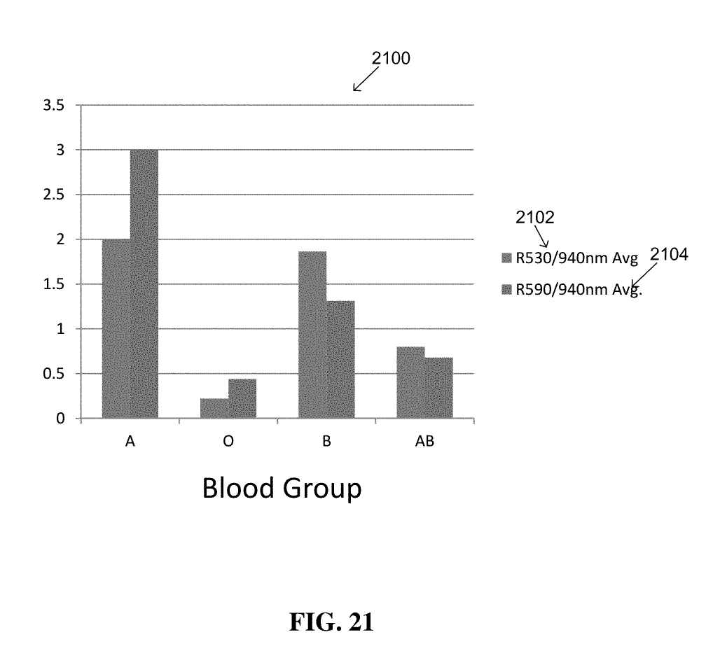

FIG. 21 illustrates a schematic drawing of an embodiment of a calibration table 2100 for blood groups. The biosensor 100 may detect red blood cells and white blood cell counts due to variances in the antigen groups on the surface of the red blood cells. The calibration table or blood group reference table 2100 includes an expected or know range of average values for R ratios for a plurality of the blood groups. The blood group reference table 2100 illustrates expected values for a plurality of blood group indicators for each blood group A, O, B, and AB. In this embodiment, the blood type indicators for the patient include an average R_{530/940} value 2102 and an average R_{530/940} value 2104. The expected average values for the blood group indicators of R_{530/940} ratio 2102 and R_{590/940} ratio 2104 are shown for each of the blood groups A, O, B and AB.

The measured average R_{530/940} value and R_{530/940} value may be compared to the blood group reference table 2100. Though the RH+ and RH− types are not shown in this blood group reference table 2100, a calibration graph or table may be used to determine the RH+ and RH− types of each Blood Groups A, B, AB and O. For example, the blood group A, B, AB and 0 may first be determined and then the RH+ and RH− types determined using the same or different blood type indicators. In another embodiment, the blood group A, B, AB and O and RH+and RH− type may be determined using a same calibration table and blood type indicators. For example, values of the R ratio at 535 nm/940 nm may be used to detect either Rh+ or Rh−.

In another embodiment, though two blood type indicators are illustrated herein, three or more blood type indicators may be used to determine the blood type or a single blood type indicator may be used to determine the blood type. For example, a first blood type indicator may be determined and compared with the blood group reference table 2100. If the first blood type indicator fails to correlate with an expected value for a blood type, one or more additional blood type indicators may be obtained and compared with the blood group reference table 2100. In addition, though the blood group reference table 2100 illustrates a single expected value for each blood type indicator, the blood group reference table 2100 may indicate a range of expected values for one or more blood type indicators. The various R values indicate a presence of an antigen to identify a blood group of A, B, O or AB using the plurality of spectral responses. The biosensor 100 may use the same R values or different R values to determine a presence of another antigen within a blood group to identify an RH factor using the plurality of spectral responses. The biosensor 100 may thus be configured to determine a blood group A, B, O, AB and RH+ and RH− using one or more blood type indicators and a blood group reference table. A blood type indicator is obtained using values of an R ratio over a sample window. A different R ratio and blood type indicator may be used for comparison based on the blood group (such as, A, B, O, AB).

For example, an expected range of values for a first blood type indicator derived from a first R ratio may be listed for blood group A while an expected range of values for a second blood type indicator derived from a second different R ratio may be listed for blood group O. The biosensor 100 may obtain the first and/or second blood type indicators in series or parallel and use the calibration table to determine the blood type. The various calibration tables, curves or other correlations may be stored in the calibration database 1200. For example, the calibration database 1200 includes a correlation of values or ranges of the one or more blood group indicators to blood group or blood type. The calibration database 1200 may be determined by obtaining the blood group indicator for a sample general population for each known blood type.

The biosensor may thus determine a first ratio R value using a first spectral response and a second spectral response of the spectral responses, wherein the first ratio R value is determined from a ratio of alternating current (AC) signal components in the first spectral response and the second spectral response and varies based on one or more of a plurality of types of antigens on surfaces of red blood cells in a blood stream of the user; access a calibration database stored in a memory that associates predetermined ratio R values to the plurality of types of antigens on the surfaces of red blood cells; and identify the blood type of the patient using the first ratio R value and the calibration database.

One or more steps of the blood type identification process may be performed by a neural network or machine learning algorithm as described in more detail herein.

The spectral differences of the antigens present on the surface of red blood cells in different blood types affects the quality of the PPG signal. For example, the reflectance of different types of surfaces of the red blood cells affects the scattering of light transmitted from the PPG circuit 110. These differences in signal quality are measurable, especially in a reflectance PPG signal (vs. a transmissive PPG signal) due to the differing light scattering properties. In known solutions, an automatic gain filter or other digital signal processing compensates for the different qualities of the PPG signal.

However, when a uniform gain or filter is applied to the PPG signal from patients of different blood types, the differences in the signal strength and qualities of the PPG signal may be measured and used to determine the blood type. The different blood groups have different optical properties due to variances in the antigen groups on the surface of the red blood cells. Thus, the quality of the PPG signal quality is affected by the type of blood group due to the different antigens on the surface of the RBCs. Various parameters that measure signal quality or signal strength of the PPG signal may be determined and compared to predetermined values to determine the blood type. These differences in PPG signal quality are preferably determined at a similar gain or amplification.

There are various signal quality parameters that may be implemented to compare the differences in signal quality and strength of the PPG signal across blood types. For example, using a similar gain or amplification and other filtering or signal processing, the cross-correlation and auto-correlation of PPG signals, may be measured to determine different blood types. Other signal quality parameters may also be implemented to determine different blood types, such as a signal to noise ratio (SNR), skewness index (S_{SQI}), a kurtosis index (K_{SQI}), entropy (E_{SQI}), relative power, or other indices of signal quality or strength. An example of types of signal quality parameters that may be implemented herein are described in, Elgendi, Mohamed, “Optimal Signal Quality Index for Photoplethysmogram Signals.” Ed. Gou-Jen Wang. Bioengineering 3.4 (2016): 21, which is hereby incorporated by reference herein. The various signal quality parameters measure the signal quality and/or signal strength of the PPG signal. A signal quality parameter may be implemented as another blood type indicator.

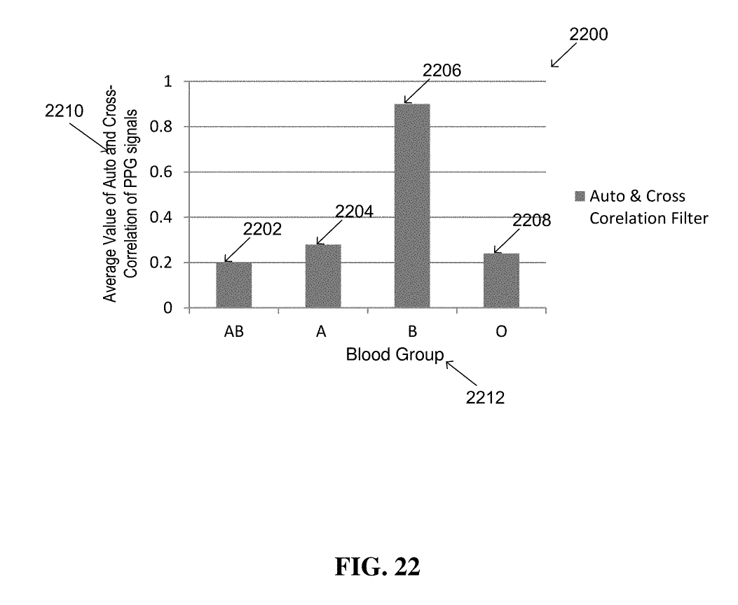

FIG. 22 illustrates a schematic graph 2200 of an embodiment of predetermined signal quality parameters for obtaining a blood type in a patient. In this graph 2200, the average values 2210 of the auto and cross-correlations of PPG signals from various blood types 2212 is illustrated. The predetermined average values include, e.g., an approximate 0.2 average for AB Blood group 2202, an approximate 0.3 average for A blood group 2204, an approximate 0.9 average for B blood group 2206, and an approximate 0.23 average for O blood group 2208. Though the approximate averages are shown, a range of average values may be predetermined for the different blood groups. Alternatively, a mean, threshold value, or other parameter derived from the auto or cross-correlation functions may be implemented.

In use, PPG signals are obtained from a patient at a plurality of wavelengths. The state of the gain controller is changed or disabled. For example, the automatic gain control is disabled, and a uniform gain or no gain is applied to the PPG signal across patients when determining blood group. In general, the PPG signal should have no gain applied or a similar gain applied for consistent measure and comparison of signal quality for blood typing.

One or more signal quality parameters are measured using one or more PPG signals at one or more wavelengths. The signal quality parameters may relate to signal quality and/or signal strength of the PPG signal. For example, a signal to noise ratio of a PPG signal with a wavelength in an IR range may be determined. An average value of an auto-correlation of a PPG signal may be determined or a cross-correlation of two PPG signals at two different wavelengths may be determined. Other signal quality parameters may also be implemented to determine different blood types, such as a skewness index (S_{SQI}), a kurtosis index (K_{SQI}), entropy (E_{SQI}), relative power, or other indices of signal quality or strength.

Though the signal quality parameter of the PPG signal I_{AC+DC} is illustrated herein, the signal quality parameter may be measured from an isolated I_{AC} component of the PPG signal. For example, melatonin or other skin tone differences may affect the I_{DC} component of the PPG signal, but the I_{AC} component reflects the pulsating volume of arterial or venous blood. The signal quality parameter of the isolated I_{AC} component of the PPG signal may thus be used to determine a blood type as well.

The one or more measured signal quality parameters are compared to predetermined signal quality parameters for the one or more blood groups. For example, the calibration database 1200 may be accessed that associates values of predetermined signal quality parameters to a plurality of blood groups (e.g., types of antigens on surfaces of red blood cells). Based on the comparison, a blood group is obtained for the user. The blood group may be determined based on a single comparison or multiple comparisons.

Upon completion of the blood typing process, the state of the automatic gain controller may be changed or enabled. For example, the automatic gain controller or other varied gain may be applied to the PPG signal for determination of other patient vitals.

The blood type of the patient affects the baseline color of the pulsatile blood flow. Thus, a calibration database 1200 may include a baseline color of pulsatile blood flow, e.g. measured at one or more wavelengths, of different sample populations having different blood types. For example, a baseline color measured at one or more wavelengths may be stored for each of a sample population having A blood group, B blood group, AB blood group, and O blood group.

### Embodiment—Detection of White Blood Cells and Infection

The biosensor 100 may detect concentration levels or counts of white blood cells in blood flow. For example, the biosensor 100 may detect the various types of white blood cells using the spectral response of one or more wavelengths, e.g. one or more wavelengths with high absorption coefficients of certain white blood cells, as shown in Table 4 below.

TABLE 4
Detection of White Blood Cells

| White Blood Cell | | | Spectral Absorption |
|---|---|---|---|
| Type | Diameter | Color | Wavelengths |
| Neutrophil | 10-12 um | Pink - Red, | Red - 660 nm |
| | | Blue, White | Blue - 470 nm |
| | | | Green - 580 nm |
| Eosinophil | 10-12 um | Pink | 660 nm, 470 nm, |
| | | Orange | 580 nm 600 nm |
| Basophil | 12-15 um | Blue | 470 nm |
| Lymphocyte | 7-15 um | | 633 nm |
| Monocyte | 15-30 um | | 580 nm |

The biosensor 100 may detect a color or color change of the blood due to an increase or decrease in white blood cells using one or more wavelengths described in Table 4 or in a range of +/−15 nm around such wavelengths. The biosensor 100 may determine an L value at the wavelength or determine an R value using the wavelength and another wavelength with a low absorption coefficient for the type of white blood cell (such as 940 nm). Based on the detected color or change in color of the blood flow, the biosensor 100 may output an alert. For example, when the color or change in color exceeds a predetermined threshold indicating a high level of WBCs, the biosensor 100 may output a visible or audible indicator.

In one embodiment, the biosensor 100 monitors the color of the blood flow. When it detects a change in the color indicating an increase in white blood cells, the biosensor 100 determines whether this color change meets a predetermined threshold indicating an elevated WBC level and/or presence of an infection. The predetermined threshold may include a color scale and/or length of time of color change. When the color change reaches the predetermined threshold, the biosensor 100 transmits or displays an alert to indicate the elevated WBC level and/or presence of an infection.

In another embodiment, the biosensor 100 may detect white blood cells from patterns in spectral responses at one or more wavelengths. Due to the larger size of the white blood cells than the size of red blood cells, the presence of white blood cells in the blood affects the width and shape of a spectral response.

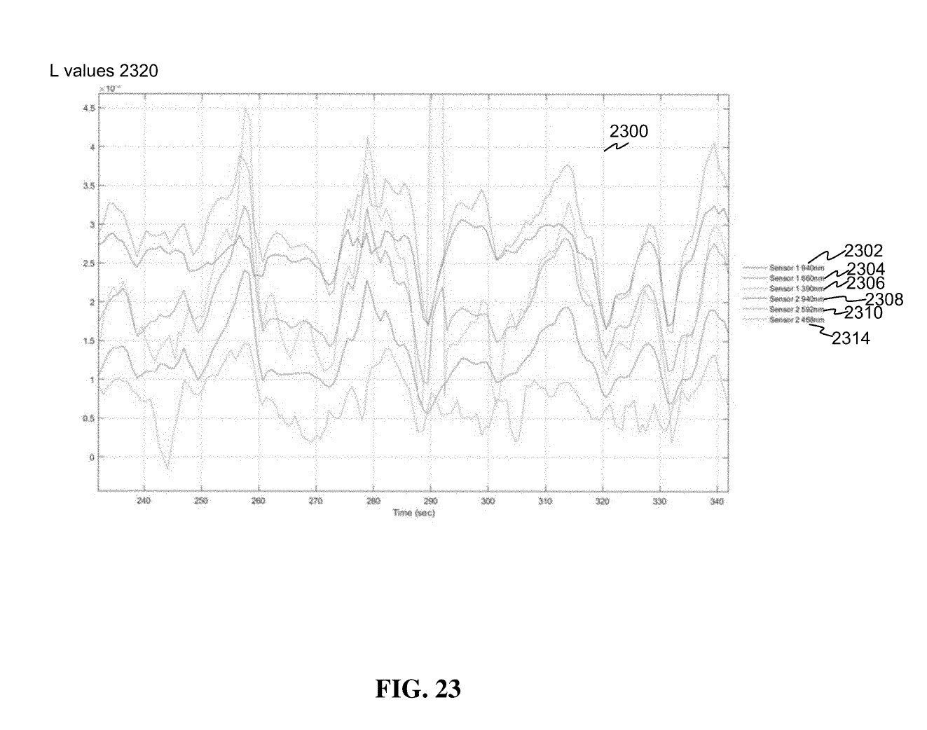

FIG. 23 illustrates an exemplary graph 2300 of spectral responses of a plurality of wavelengths from clinical data using the biosensor 100. In this embodiment, the spectral response of a plurality of wavelengths was measured using the biosensor 100 over a measurement period of almost 600 seconds or approximately 10 minutes. The graph 2300 illustrates the L values calculated from the spectral response for a first wavelength 2302 of approximately 940 nm, the spectral response for a second wavelength 2304 of approximately 660 nm and the spectral response for a third wavelength 2306 of approximately 390 nm obtained from a first biosensor 150 measuring reflected light from a first fingertip of a patient. The graph further illustrates the spectral response for a fourth wavelength 2310 of approximately 592 nm and a fifth wavelength 2314 of approximately 468 nm and the spectral response 1408 again at 940 nm obtained from a second biosensor measuring reflected light from a second fingertip of a patient. The spectral responses are temporally aligned using the systolic and diastolic points. Though two biosensors were used to obtain the spectral responses in this clinical trial, a single biosensor 150 may also be configured to obtain the spectral responses of the plurality of wavelengths.

Due to the size of white blood cells, the presence of the white blood cells in the blood affects the spectral width and shape of a spectral response at one or more wavelengths. In one aspect, from L values 2320 shown for the spectral response at 660 nm, the width and shape of the spectral response is affected by the presence of white blood cells. For example, the width and shape of L_{660 nm} between 250 and 270 seconds has a different shape and width of L_{660 nm} between 300 and 320 seconds in the graph 2300. The differences in the width and shape of the spectral response may be used to determine a concentration level of white blood cells or change in concentration level of white blood cells in the blood.

In another example, the spectral responses may be used to determine a presence of infection from a level of neutrophils or neutrophilic white blood cells in blood flow. The concentration of neutrophils increases in the presence of an infection. The neutrophil particles have a different color and size from red blood cells. The biosensor 100 may determine an increase in concentration of neutrophil cells in response to a change in color of the blood or in response to a difference from a baseline color. In addition, the biosensor 100 may determine an increase in concentration of neutrophil cells in response to a change in a pattern of the spectral response (L value and/or R value) due to a change in size of particles in the blood. The biosensor 150 may use a combination of both a change in color and change in a pattern of the spectral response (L value and/or R value) to determine a concentration of neutrophils.

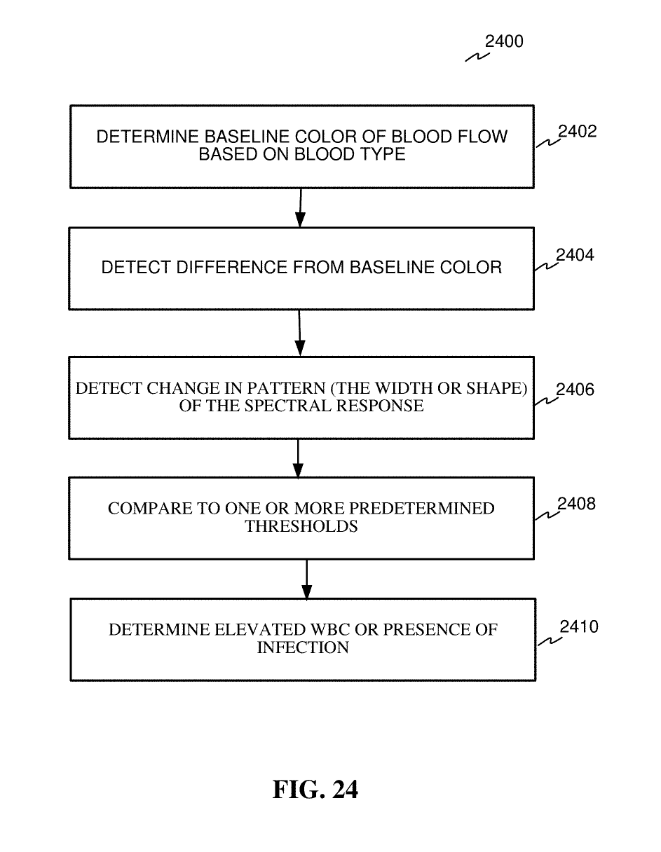

FIG. 24 illustrates a logical flow diagram of a method 2400 for detecting a presence of infection using a level of white blood cells in blood flow. The biosensor 100 determines a baseline color of blood flow at 2402. The baseline colors of blood flow may be stored in a calibration database 1200 for each of a plurality of blood types. The blood type of the patient is then determined, and the baseline color of blood flow for that blood type is obtained, e.g. from the calibration database 1200. The baseline color of the blood flow for an individual patient may also be obtained by measuring blood flow of the patient when healthy, e.g. prior to a surgery, infection or injury.

The biosensor 100 monitors the color of the blood flow at one or more wavelengths as shown in Table 4 to detect a difference in the color of the blood flow from the baseline color at 2404. The biosensor 100 may also detect a change in a pattern of the spectral response, such as the width and shape, of the spectral response indicating a presence of white blood cells at 2406.

The biosensor 100 determines whether the change in pattern and/or difference in color meets a predetermined threshold at 2408. The predetermined threshold may be set to indicate an elevated level of white blood cells. For example, the predetermined threshold may include a percentage change in color and/or length of time indicating elevated levels of WBCs or RBCs at 2410. The change in color or pattern may be used to determine a concentration level of white blood cells or change in concentration level of white blood cells in the blood. For example, a difference in color from the baseline may be correlated to a white blood cell count or percentage increase in white blood cells (or RBC level). The biosensor 100 may then generate an indication of the level of white blood cells or red blood cells in blood flow based on the comparison.

In another example, the predetermined threshold may be set to a threshold indicating a presence of an infection at 2410. For example, the predetermined threshold may include a color scale and/or length of time of color change indicating a presence of an infection. The predetermined threshold may include a change in the width or shape of the spectral response due to white blood cells. The biosensor 100 may then indicate an infection, wherein the type of infection may include a staph infection or a sepsis infection. For example, the type of infection may include an afebrile sepsis, febrile sepsis, hemolytic staph infection or non-hemolytic staph infection. The biosensor 100 may measure other parameters to identify the type of infection, such as hemolysis, NO levels, liver enzyme levels, vasodilation, heart rate, respiration rate and skin temperature.

### Embodiment—Detection of Infection using Measurements of Vasodilation

In an embodiment, the biosensor 100 detects an infection or potential infection using measurements of vasodilation. Vasodilation is the widening of blood vessels. It results from relaxation of smooth muscle cells within the vessel walls, in particular in the large veins, large arteries, and smaller arterioles. The process is the opposite of vasoconstriction, which is the narrowing of blood vessels due to constriction of the smooth muscle cells within the vessels walls. The vascular endothelium is crucially involved in the fundamental regulation of blood flow matching demand and supply of tissue. After transient ischemia, arterial inflow increases. As a response to increased shear forces during reactive hyperemia, healthy arteries dilate via release of NO or other endothelium-derived vasoactive substances. This endothelium-dependent flow-mediated vasodilation (FMD) is impaired in atherosclerosis.

The capacity of blood vessels to respond to physical and chemical stimuli in the lumen confers the ability to self-regulate tone and to adjust blood flow and distribution in response to changes in the local environment. Many blood vessels respond to an increase in flow, or more precisely shear stress, by dilating. This phenomenon is designated flow-mediated vasodilation (FMD). A principal mediator of FMD is endothelium-derived NO—an example of an EDRF.

An infection in tissue, such as around a wound site, creates increased blood flow in the tissue due to vasodilation. The inflamed, swollen tissue exhibits an increase in vasodilation. Additional details on detection of vasodilation are described in U.S. patent application Ser. No. 16/172,661, filed Oct. 26, 2018, entitled, “System and Method of a Biosensor for Detection of Vasodilation,” which is hereby incorporated by reference herein.

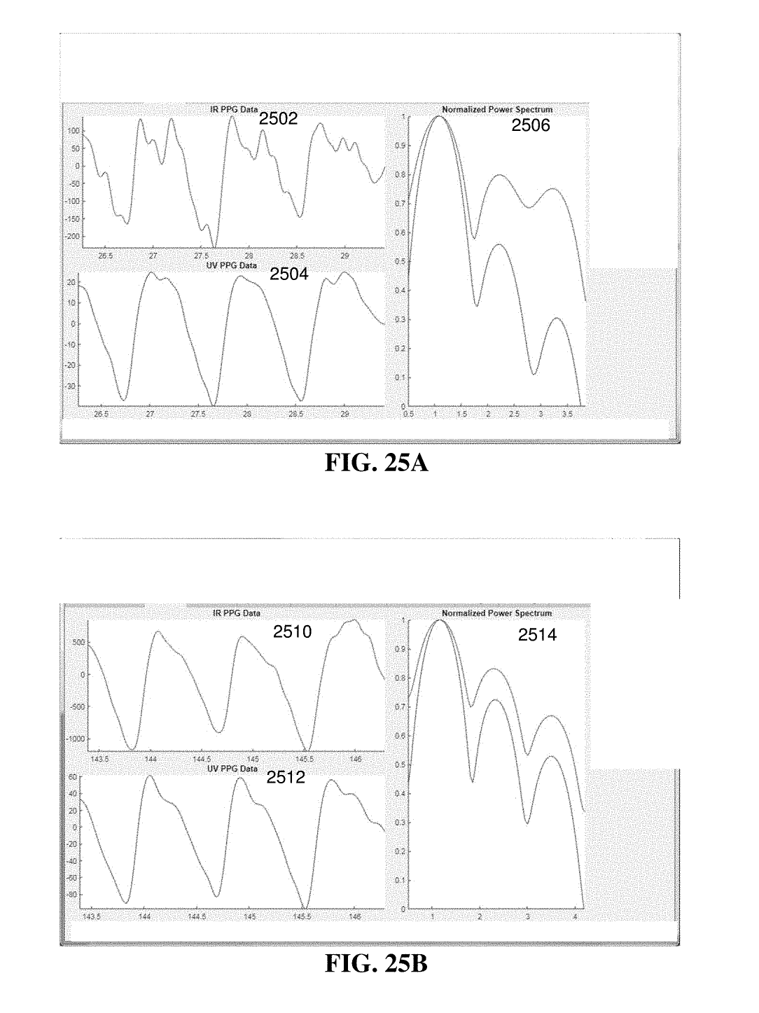

FIG. 25A illustrates a schematic diagram of graphs of PPG signals detected from normal tissue. In this embodiment, a biosensor 100 is positioned on a forearm over normal tissue. Graph 2502 illustrates a PPG signal at an IR wavelength, such as 880 nm or 940 nm. The Graph 2504 illustrates a PPG signal at a UV range, such as 395 nm or 405 nm. The PPG signals reflect the pulse pressure wave due to the cardiac cycle. Due to the increased penetration of the IR wavelength in the tissue, the PPG signal at the IR wavelength has a different pulse shape than the PPG signal at the UV wavelength. Graph 2506 illustrates a normalized power spectrum of the PPG signals at the UV and IR wavelengths. Due to the increased penetration and greater blood volume detected, the PPG signal at the IR wavelength has an increased normalized power spectrum.

FIG. 25B illustrates a schematic diagram of graphs of PPG signals detected from tissue after an impact. Graph 2502 illustrates a PPG signal at an IR wavelength, such as 880 nm or 940 nm. The Graph 2504 illustrates a PPG signal at a UV range, such as 395 nm or 405 nm. In this embodiment, the biosensor 100 is positioned on the forearm over the same tissue after an impact to the forearm. The forearm has turned red and swollen due to increased blood flow to the tissue but incurs no abrasions or bruising. Due to the increased blood flow to the surface of the tissue, e.g. due to vasodilation of the vessels in the tissue, the PPG signals at the IR wavelength and UV wavelength have a more similar pulse shape and signal to noise ratio. In addition, the normalized power spectrum of the PPG signals in Graph 2514 are more similar as well due to the increase of blood flow at the surface of the tissue. When similar blood flow is detected at the different levels of tissue, the PPG signals at UV and IR wavelengths have a more similar pulse shape and power spectrum. This similarity in the PPG signals at the different wavelengths indicates an increase of blood flow in the surface tissue due to vasodilation from injury or infection.

The biosensor 100 may thus detect vasodilation and an infection at a wound site by comparing the pulse shape and normalized power spectrum of PPG signals in different spectrums. The infection creates increased blood flow, swelling and vasodilation in the tissue around the wound site. The biosensor 100 may detect this change in the optical absorption properties of the tissue and the presence of vasodilation and infection or possible infection.

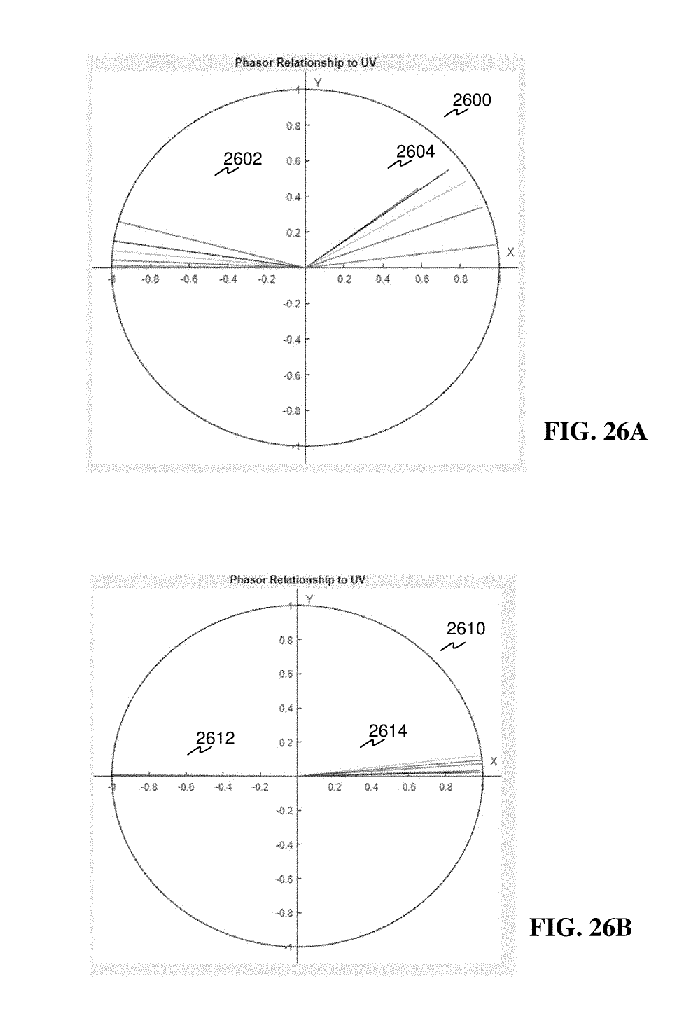

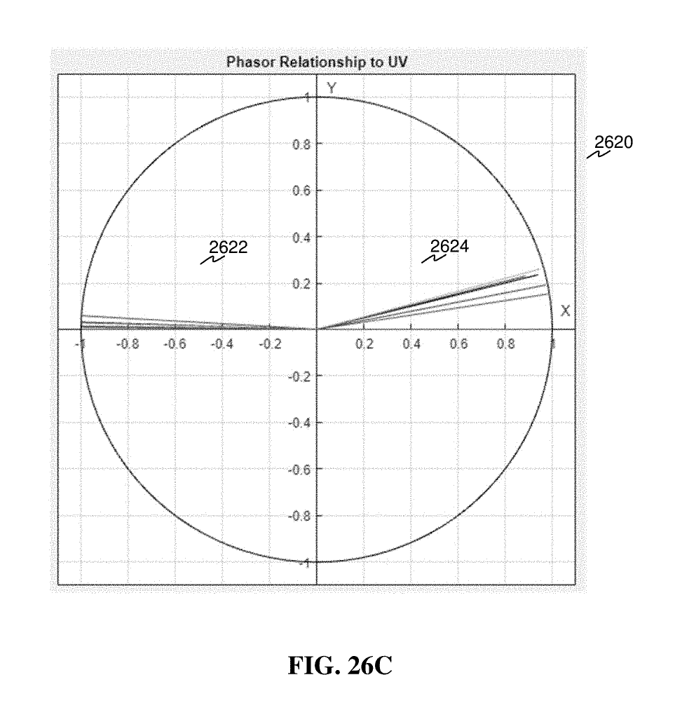

FIG. 26A illustrates a schematic diagram 2600 of a phasor relationship between PPG signals detected from normal tissue. The PPG signals were detected by the biosensor 100 positioned on a forearm over normal tissue as in FIG. 25A. In this embodiment, the biosensor 100 obtained the PPG signals at a plurality of wavelengths across UV, visible and IR spectrums. For example, the PPG signals may be at wavelengths of approximately Infrared: 940 nanometers, Red: 630 nanometers, Yellow: 590 nanometers, Green: 525 nanometers, Blue: 465 nanometers, UV 405 nanometers. These wavelengths are exemplary and other wavelengths may be implemented.

In Graph 2602, the PPG signals in the UV, visible and IR spectrums are processed using a linear fit model and transformed into vectors that represent a phase offset between the respective PPG signals. The angle or phase of the vectors in Graph 2902 are a representation of the phase offset or timing differences between the PPG signals in the different spectrums in normal tissue. In this example, the phase offset of the PPG signals is measured from the PPG signal at the UV wavelength. As such, the PPG signal at the UV wavelength is shown with no phase offset. The magnitude of the vectors is each scaled to a same value. As shown in Graph 2602, in the normal tissue, the PPG signals exhibit only a slight difference in phase or timing. This indicates that the tissue has normal circulation and nominal vasodilation is occurring in the tissue.

In Graph 2604, the PPG signals in the UV, visible and IR spectrums are processed using a Hilbert transformation into vectors, wherein a vector represents a difference in pulse shape and timing offset between the PPG signals. The Hilbert transformation is just one of many models that can be used to compare the pulse shape and temporal relationship between PPG signals and reduce the data to a vector or set of vectors. For example, the magnitude of the vectors represents a difference in the pulse shapes between the PPG signals. The phase of the vectors represents the temporal or timing differences between the PPG signals. These differences may be represented by similarity values, e.g. differences in pulse shape or temporal differences in the PPG signals. As shown in Graph 2604, in the normal tissue, the PPG signals exhibit differences in the magnitude of the vectors, and there is a greater difference in the phase offsets, e.g. the PPG signals have similar pulse shapes, but there are slight temporal differences between the signals. This indicates that the tissue has normal circulation and nominal vasodilation is occurring in the tissue.

FIG. 26B illustrates a schematic diagram 2610 of graphs of PPG signals detected from tissue after an impact. In this embodiment, the biosensor 100 is positioned on the forearm over the same tissue after an impact to the forearm. The forearm has turned red and swollen due to increased blood flow to the tissue but incurs no abrasions or bruising. Due to the increased blood flow to the surface of the tissue, e.g. due to vasodilation of the vessels in the tissue, the PPG signals as represented in Graph 2612 have a decreased phase offset from normal tissue.

As such, the phase offset between two or more of the PPG signals in different spectrums, or having different depths of penetration of tissue, is measured. The phase offset may be used to determine presence of vasodilation/vasoconstriction or a problem with circulation in the tissue. The phase offset may be mapped to a level of vasodilation, e.g. using a calibration table or function. In addition, a phase offset may be used to determine a blood circulation level in the tissue, e.g. an abnormal circulation or a normal circulation. The abnormal circulation may include a high or low circulation level. A phase offset lower than a predetermined range (e.g. from a normal tissue measurement) may indicate a blood circulation problem in the tissue, e.g. that indicates increased blood flow due an infection or injury or that indicates a low circulation level. The predetermined range may be determined from testing healthy tissue of the patient or from testing healthy tissue of a general sample population.

The phase offset at one or more tissue sites may also be used to determine a systemic circulation level. For example, a low circulation level in several tissue sites of each leg of a patient may be used to determine a systemic circulation problem. In addition, NO levels, SpO₂, and/or skin temperature may also be used to determine a systemic circulation problem.

Graph 2614 illustrates the PPG signals as vectors, wherein the vectors represent a difference in pulse shape and phase offset of the PPG signals. For example, the magnitude of a vector represents a difference in the pulse shape of the PPG signal from a baseline heart rate PPG signal or between the PPG signals. The phase of a vector represents the temporal or timing differences between the PPG signal and a baseline heart rate PPG signal or between the PPG signals. These differences may be represented by a correlation or similarity value, e.g. differences in amplitude pulse shape and/or temporal differences in the PPG signal. When blood flow is increased to the tissue, the PPG signals at UV and IR wavelengths exhibit a lower variance in pulse shape and a higher correlation value. This decrease in the difference in the pulse shape of the PPG signals at the different wavelengths indicates an increase of blood flow in the surface tissue due to vasodilation from the injury.

As such, the extent or magnitude of the correlation in pulse shape between two or more of the PPG signals in different spectrums, or having different depths of penetration of tissue, may be used to determine presence of vasodilation/vasoconstriction or a circulation level in the tissue. The injury or infection creates increased blood flow, swelling and vasodilation in the tissue around the wound site. The biosensor 100 may detect this change in blood flow or vasodilation, and so detect a risk of infection or possible injury. A difference between pulse shapes or phase offset outside a normal range (e.g. from a normal tissue measurement) may indicate a blood circulation problem in the tissue, e.g. that indicates increased blood flow due to an infection or injury or that indicates high circulation. The correlation value may thus also be used to determine a blood circulation level in the tissue, e.g. a normal circulation or abnormal circulation (lower than normal or higher than normal). In addition, the correlation values of the PPG signals may be mapped to a level of vasodilation, e.g. using a calibration table or function.

FIG. 26C illustrates a schematic diagram 2620 of graphs of PPG signals detected from tissue healing after an impact. In this embodiment, the biosensor 100 is positioned on the forearm as in FIG. 26B, and the PPG signals are obtained after the tissue has partially healed. The forearm has returned to a more normal skin tone and swelling has decreased. The vectors in Graph 2622 are a representation of the phase or timing offset between the PPG signals in the different spectrums in the healing tissue. In the healing tissue, the PPG signals have more phase offset than in the recently damaged tissue but still less than the normal tissue. This indicates that less vasodilation is occurring in the healing tissue than the injured tissue but more than in the normal tissue, and blood circulation is returning to normal as the tissue heals.

In Graph 2624, the PPG signals in the UV, visible and IR spectrums are processed using a Hilbert transformation into vectors that represent a pulse shape and phase offset between the PPG signals. The Hilbert transformation is just one of many models that can be used to compare the pulse shape and temporal relationship between PPG signals and reduce the data to a vector or set of vectors. The magnitude of the vectors represents a magnitude or extent of the difference in pulse shape and phase represents the phase offset of the PPG signal from other PPG signals. The differences in pulse shape and phase offset between the PPG signals have increased in the healing tissue as compared to the injured tissue. This indicates that less vasodilation is occurring in the healing tissue than in the injured tissue but more than in the normal tissue and blood circulation is returning to normal as the tissue heals.

The PPG signals may thus be used to determine a circulation level in skin tissue, detect an injury or infection in tissue and a level of vasodilation. The PPG signals obtained at one or more tissue sites may also be used to determine a systemic circulation level. For example, a low circulation level in several tissue sites of each leg of a patient may be used to determine a systemic circulation problem. In addition, NO levels, SpO₂, and/or skin temperature may also be used to determine a systemic circulation problem.

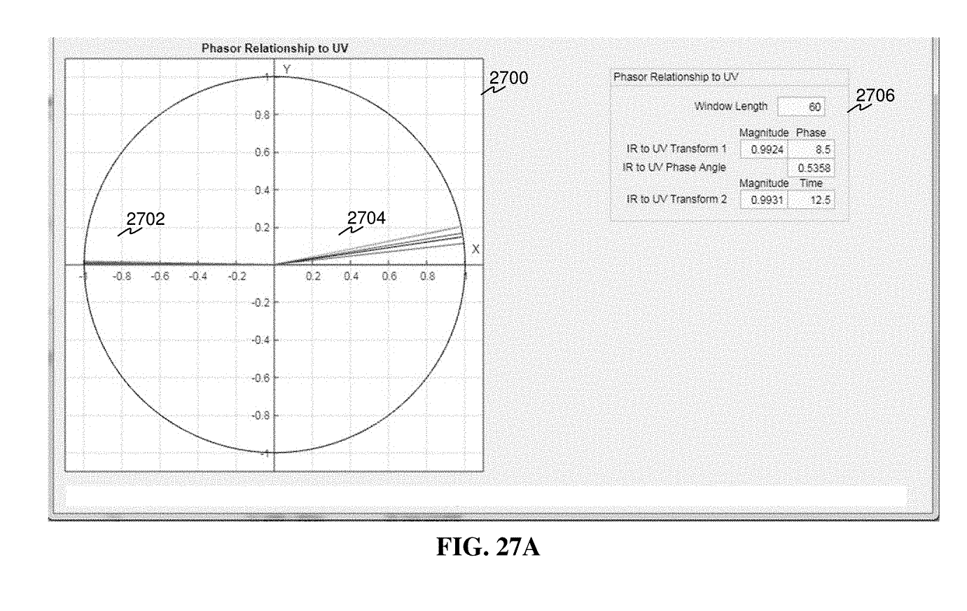

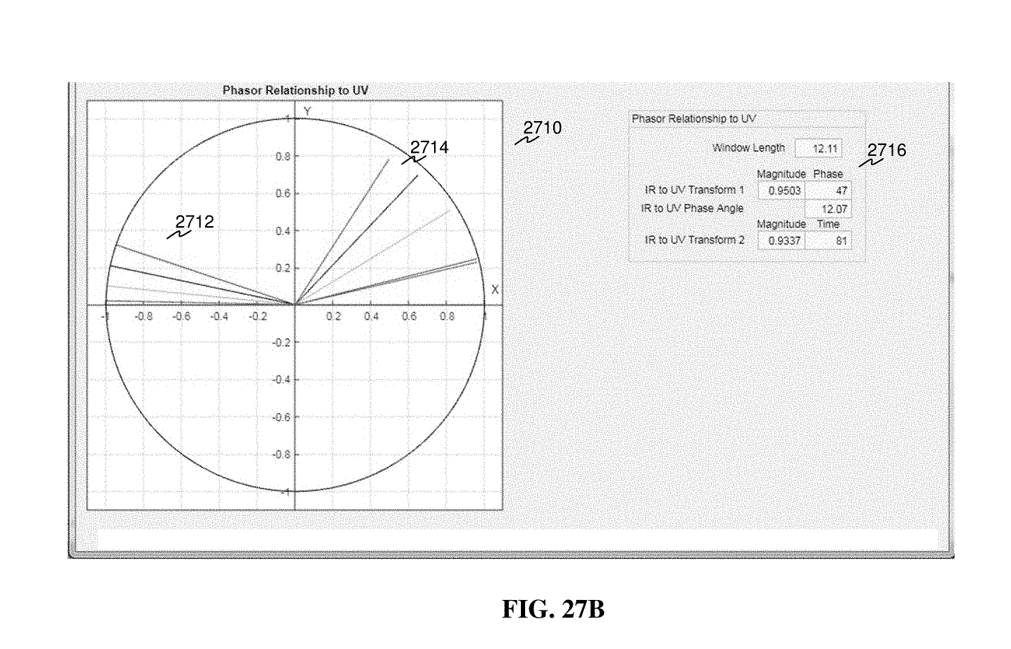

FIG. 27A illustrates a schematic diagram 2700 of a phasor relationship of PPG signals detected from normal tissue. In this embodiment, the biosensor 100 obtained PPG signals at a plurality of wavelengths across UV, visible and IR spectrums. For example, the PPG signals may be at wavelengths of approximately Infrared: 940 nanometers, Red: 630 nanometers, Yellow: 590 nanometers, Green: 525 nanometers, Blue: 465 nanometers, UV 405 nanometers. These wavelengths are exemplary and other wavelengths in the UV, visible or IR spectrums may be implemented. The PPG signals were detected by the biosensor 100 positioned on a fingertip having normal tissue and temperature.

In Graph 2702, the PPG signals in the UV, visible and IR spectrums are processed using a linear fit model and transformed into vectors with an angle that represent a phase offset of the respective PPG signal. The magnitude of the vectors is each scaled to a same value. The vectors in Graph 2702 are thus a representation of the phase or timing differences between the PPG signals. In the normal tissue, the PPG signals have little to no difference in phase or timing. This indicates that little to no vasodilation or vasoconstriction is occurring in the normal tissue at the fingertip and blood circulation is normal.

In Graph 2704, the PPG signals in the UV, visible and IR spectrums are processed using a Hilbert transformation into vectors that represent the similarity in pulse shape and phase offset between the PPG signals. The Hilbert transformation is just one of many models that can be used to compare the pulse shape and temporal relationship between PPG signals and reduce the data to a vector or set of vectors. For example, the magnitude of the vectors represents a difference in the pulse shapes between the PPG signals. The phase of the vectors represents the temporal or timing differences between the PPG signals. These differences may be represented by correlation values, e.g. differences in pulse shape or temporal differences in the PPG signals. A chart 2706 provides numerical values for the magnitude and phase of the Hilbert Data in Graph 2704 in a first line and a phase angle for the linear fit model of Graph 3002 in a second line. A cross correlation value of the PPG signals at an IR and UV wavelength is shown in a third line, this correlation value is another model for comparing the pulse shape and temporal relationship between PPG signals and reducing the data to a vector or set of vectors.

FIG. 27B illustrates a schematic diagram 2710 of a phasor relationship of PPG signals detected from tissue at sub-normal temperature. In this embodiment, the biosensor 100 obtained the PPG signals at wavelengths of approximately Infrared: 940 nanometers, Red: 630 nanometers, Yellow: 590 nanometers, Green: 525 nanometers, Blue: 465 nanometers, UV 405 nanometers. These wavelengths are exemplary and other wavelengths in the UV, visible or IR spectrums may be implemented. The PPG signals were detected by the biosensor 100 positioned on the fingertip as in FIG. 27A after a temperature of the finger was lowered. The finger was submerged in ice water for a period of thirty seconds to lower the temperature of the surface tissue in the finger.

In Graph 2712, the PPG signals in the UV, visible and IR spectrums are processed using a linear fit model and transformed into vectors with an angle that represent a phase offset of the respective PPG signal. The magnitude of the vectors is each scaled to a same value. The vectors in Graph 2712 are thus a representation of the phase or timing offset between the PPG signals in the different spectrums in the tissue having a subnormal temperature. In this tissue, the PPG signals have a greater difference in phase or timing. This indicates that blood flow in the colder tissue near the surface is decreased, e.g. due to vasoconstriction, a low blood circulation level.

As such, the phase offset between two or more of the PPG signals in different spectrums, or having different depths of penetration of tissue, is measured. The phase offset may be used to determine presence of vasodilation/vasoconstriction or a circulation level in the tissue. The phase offset may be mapped or correlated to a level of vasodilation, e.g. using a calibration table or function. In addition, a phase offset exceeding a predetermined threshold (e.g. from a normal tissue measurement) may indicate a blood circulation problem in the tissue, e.g. that indicates increased blood flow due an infection or injury or that indicates low circulation.

In Graph 2714, the PPG signals in the UV, visible and IR spectrums are processed using a Hilbert transformation into vectors that represent the similarity in pulse shape and phase offset been the PPG signals. The Hilbert transformation is just one of many models that can be used to compare the pulse shape and temporal relationship between PPG signals and reduce the data to a vector or set of vectors. For example, the magnitude of the vectors represents a difference in the pulse shapes between the PPG signals. The phase of the vectors represents the temporal or timing differences between the PPG signals. These differences may be represented by correlation values, e.g. differences in pulse shape or temporal differences in the PPG signals. Again, in this tissue, the PPG signals have a greater difference or lower correlations in pulse shape. This indicates that blood flow in the colder surface tissue is decreased, e.g. due to vasoconstriction, and a low circulation level.

A chart 2716 provides numerical values for the magnitude and phase of the Hilbert Data in Graph 2714 in a first line and a phase angle for the linear fit model of Graph 2712 in a second line. A cross correlation value of the PPG signals at the IR and UV wavelength is shown in a third line, this correlation value is another model for comparing the pulse shape and temporal relationship between PPG signals and reducing the data to a vector or set of vectors.

The biosensor 100 may detect this change in blood flow or vasoconstriction, and so the presence of infection or possible injury. A correlation value that represents a difference between pulse shapes and/or temporal differences of different PPG signals exceeding or lower than predetermined thresholds (e.g. from a normal tissue measurement) may indicate an abnormal circulation in the tissue, e.g. an increased blood flow due an infection or injury or a low circulation level. In addition, the correlation value may be mapped to a level of vasodilation, e.g. using a calibration table or function.

The PPG signals may thus be used to detect a circulation level, infection, arterial stiffness, etc. One or more parameters derived using the PPG signals may be used to determine a period of vasodilation/vasoconstriction or a level of vasodilation/vasoconstriction. A phase offset between PPG signals or a correlation value between PPG signals may be used to determine a circulation level (such as high, normal or low) or possible injury or infection.

The calibration database 1200 may includes one or more calibration tables for mapping parameters obtained from PPG signals to a level of vasodilation. For example, values of phase offset between PPG signals may be mapped to corresponding levels of vasodilation. In another example, low frequency components I_{DC} values may be mapped to corresponding levels of vasodilation. In another example, values of correlations between PPG signals may be mapped to corresponding levels of vasodilation.

In one or more aspects herein, a processing module or circuit includes at least one processing device, such as a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. A memory is a non-transitory memory device and may be an internal memory or an external memory, and the memory may be a single memory device or a plurality of memory devices. The memory may be a read-only memory, random access memory, volatile memory, non-volatile memory, static memory, dynamic memory, flash memory, cache memory, and/or any non-transitory memory device that stores digital information.

As may be used herein, the term “operable to” or “configurable to” indicates that an element includes one or more of circuits, instructions, modules, data, input(s), output(s), etc., to perform one or more of the described or necessary corresponding functions and may further include inferred coupling to one or more other items to perform the described or necessary corresponding functions. As may also be used herein, the term(s) “coupled”, “coupled to”, “connected to” and/or “connecting” or “interconnecting” includes direct connection or link between nodes/devices and/or indirect connection between nodes/devices via an intervening item (e.g., an item includes, but is not limited to, a component, an element, a circuit, a module, a node, device, network element, etc.). As may further be used herein, inferred connections (i.e., where one element is connected to another element by inference) includes direct and indirect connection between two items in the same manner as “connected to”.

As may be used herein, the terms “substantially” and “approximately” provides an industry-accepted tolerance for its corresponding term and/or relativity between items. Such an industry-accepted tolerance ranges from less than one percent to fifty percent and corresponds to, but is not limited to, frequencies, wavelengths, component values, integrated circuit process variations, temperature variations, rise and fall times, and/or thermal noise. Such relativity between items ranges from a difference of a few percent to magnitude differences.

Note that the aspects of the present disclosure may be described herein as a process that is depicted as a schematic, a flowchart, a flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed. A process may correspond to a method, a function, a procedure, a subroutine, a subprogram, etc. When a process corresponds to a function, its termination corresponds to a return of the function to the calling function or the main function.

The various features of the disclosure described herein can be implemented in different systems and devices without departing from the disclosure. It should be noted that the foregoing aspects of the disclosure are merely examples and are not to be construed as limiting the disclosure. The description of the aspects of the present disclosure is intended to be illustrative, and not to limit the scope of the claims. As such, the present teachings can be readily applied to other types of apparatuses and many alternatives, modifications, and variations will be apparent to those skilled in the art.

In the foregoing specification, certain representative aspects of the invention have been described with reference to specific examples. Various modifications and changes may be made, however, without departing from the scope of the present invention as set forth in the claims. The specification and figures are illustrative, rather than restrictive, and modifications are intended to be included within the scope of the present invention. Accordingly, the scope of the invention should be determined by the claims and their legal equivalents rather than by merely the examples described. For example, the components and/or elements recited in any apparatus claims may be assembled or otherwise operationally configured in a variety of permutations and are accordingly not limited to the specific configuration recited in the claims.

Furthermore, certain benefits, other advantages and solutions to problems have been described above with regard to particular embodiments; however, any benefit, advantage, solution to a problem, or any element that may cause any particular benefit, advantage, or solution to occur or to become more pronounced are not to be construed as critical, required, or essential features or components of any or all the claims.

As used herein, the terms “comprise,” “comprises,” “comprising,” “having,” “including,” “includes” or any variation thereof, are intended to reference a nonexclusive inclusion, such that a process, method, article, composition or apparatus that comprises a list of elements does not include only those elements recited, but may also include other elements not expressly listed or inherent to such process, method, article, composition, or apparatus. Other combinations and/or modifications of the above-described structures, arrangements, applications, proportions, elements, materials, or components used in the practice of the present invention, in addition to those not specifically recited, may be varied or otherwise particularly adapted to specific environments, manufacturing specifications, design parameters, or other operating requirements without departing from the general principles of the same.

Moreover, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is intended to be construed under the provisions of 35 U.S.C. § 112(f) as a “means-plus-function” type element, unless the element is expressly recited using the phrase “means for” or, in the case of a method claim, the element is recited using the phrase “step for.”

1. A biosensor, comprising:
a photoplethysmography (PPG) circuit configured to obtain spectral responses at one or more wavelengths from skin tissue of a patient; and
a processing circuit configured to:
obtain a blood type of the patient;
obtain a baseline color of blood flow of the patient using the blood type of the patient;
determine a color of the blood flow of the patient using one or more of the spectral responses; and
determine a difference in color of the blood flow from the baseline color of the blood flow.

2. The biosensor of claim 1, wherein the processing circuit is further configured to:
determine a change in a level of white blood cells or red blood cells in blood flow using the difference in color of the blood flow from the baseline color of the blood flow.

3. The biosensor of claim 1, wherein the processing circuit is further configured to:
determine an infection using the difference in the color of the blood flow.

4. The biosensor of claim 1, wherein the processing circuit is further configured to:
compare the difference in the color of the blood flow to a predetermined threshold; and
determine a presence of hemolysis in the blood flow based on the comparison.

5. The biosensor of claim 1, wherein the processing circuit is further configured to:
determine a level of red blood cells affected by hemolysis in the blood flow using the difference in the color of the blood flow.

6. The biosensor of claim 1, wherein the processing circuit is further configured to:
determine a measurement of a liver enzyme in the blood flow using one or more of the spectral responses; and
determine a type of infection using the measurement of the liver enzyme and the difference in the color of the blood flow.

7. The biosensor of claim 1, wherein the processing circuit is further configured to:
determine a measurement of nitric oxide (NO) in the blood flow using one or more of the spectral responses; and
determine a type of infection using the measurement of NO and the difference in the color of the blood flow.

8. The biosensor of claim 1, wherein the processing circuit is further configured to:
determine a heart rate and vasodilation using one or more of the spectral responses; and
determine the type of infection using the measurement of the heart rate, vasodilation and the difference in the color of the blood flow.

9. The biosensor of claim 1, wherein the processing circuit is further configured to obtain the blood type of the patient by:
determining a first ratio R value using a first spectral response and a second spectral response of the spectral responses, wherein the first ratio R value is determined from a ratio of alternating current (AC) signal components in the first spectral response and the second spectral response and varies based on one or more of a plurality of types of antigens on surfaces of red blood cells in a blood stream of the user;
accessing a calibration database stored in a memory that associates predetermined ratio R values to the plurality of types of antigens on the surfaces of red blood cells; and
identifying the blood type of the patient using the first ratio R value and the calibration database.

10. The biosensor of claim 1, wherein the biosensor is implemented on a disposable patch including a visible or audible indicator of an infection.

11. A biosensor, comprising:
a photoplethysmography (PPG) circuit configured to obtain spectral responses at one or more wavelengths from skin tissue of a patient; and
a processing circuit configured to:
obtain a measurement of a color of blood flow using one or more of the spectral responses;
compare the measurement of the color of blood flow to a predetermined threshold; and
generate an indication of a level of white blood cells or red blood cells in blood flow based on the comparison.

12. The biosensor of claim 11, wherein the processing circuit is configured to:
generate an indication of infection based on the level of white blood cells or red blood cells in blood flow.

13. The biosensor of claim 11, wherein the processing circuit is configured to:
determine a pattern of the one or more of the spectral responses indicating a presence of white blood cells; and
determine the level of white blood cells in the blood flow using the pattern of the one or more of the spectral responses.

14. The biosensor of claim 13, wherein the processing circuit is configured to determine the pattern of the one or more of the spectral responses indicating a presence of white blood cells by determining a change in the width and shape of the spectral response due to a size of the white blood cells.

15. The biosensor of claim 14, wherein the processing circuit is configured to determine an increase in concentration of neutrophil white blood cells in response to the color of the blood flow and the change in the pattern of the one or more of the spectral responses.

16. The biosensor of claim 11, wherein the processing circuit is further configured to:
obtain a temperature of the patient;
determine one or more of a liver enzyme level or NO level in blood flow using the one or more of the spectral responses; and
determine a type of infection using the color of the blood flow, temperature and one or more of the liver enzyme level or NO level in blood flow.

17. The biosensor of claim 16, wherein the type of infection includes: afebrile sepsis, febrile sepsis, hemolytic staph infection or non-hemolytic staph infection.

18. A biosensor, comprising:
a photoplethysmography (PPG) circuit configured to obtain spectral responses at a plurality of wavelengths from skin tissue of the patient; and
a processing circuit configured to:
determine a baseline color of blood flow of the patient using one or more of the plurality of spectral responses;
determine a change in the baseline color of blood flow using the one or more of the plurality of spectral responses; and
determine a level in blood flow of at least one of: red blood cells and white blood cells.

19. The biosensor of claim 18, wherein the processing circuit is further configured to:
compare the change in the baseline color of blood flow to a predetermined threshold; and
determine a risk of infection based on the comparison.

20. The biosensor of claim 18, wherein the processing circuit is further configured to:
determine a type of infection in blood flow using the change in the baseline color of blood flow.
